(19) United States
(12) Patent Application Publication
(10) Pub. No.: US 2014/0088114 A1
(43) Pub. Date: Mar. 27, 2014

(54) FUSED BICYCLIC KINASE INHIBITORS

(71) Applicants: Meizhong Jin, East Northport, NY (US); Mark J. Mulvihill, Dix Hills, NY (US); Arno G. Steinig, Carmel, NY (US); Jing WANG, Syosset, NY (US)

(72) Inventors: Meizhong Jin, East Northport, NY (US); Mark J. Mulvihill, Dix Hills, NY (US); Arno G. Steinig, Carmel, NY (US); Jing WANG, Syosset, NY (US)

(73) Assignee: OSI Pharmaceuticals ,LLC

(21) Appl. No.: 14/117,741

(22) PCT Filed: May 15, 2012
(86) PCT No.: PCT/US12/37866

§ 371 Date: Nov. 14, 2013

Related U.S. Application Data
(60) Provisional application No. 61/486,364, filed on May 16, 2011.

(51) Int. Cl. C07D 471/04

(52) U.S. Cl. CPC C07D 471/04
USPC 514/253.04; 546/113; 514/300; 544/362

(57) ABSTRACT

Compounds of Formula (I), pharmaceutically acceptable salts thereof, synthesis, intermediates, formulations, and methods of disease treatment therewith, including treatment of cancers, such as tumors driven at least in part by at least one of MET, RON, ALK, IR, or IGF-1R. This Abstract is not limiting of the invention.

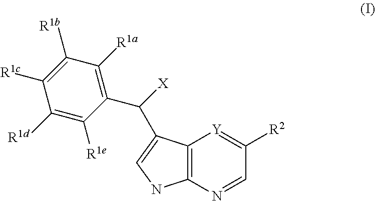

### FIELD AND BACKGROUND

The present invention pertains at least in part to cancer treatment, certain chemical compounds, and methods of treating tumors and cancers with the compounds.

RON (recepteur d'origine nantais) is a receptor tyrosine kinase that is part of the MET proto-oncogene family. It is activated by binding to its natural ligand MSP and signals via the PI3K and MAPK pathways. RON can be deregulated in cancer by mechanisms such as over-expression of the receptor and/or the presence of constitutively active splice variants. Inhibition of RON has been shown to lead to a decrease in proliferation, induction of apoptosis and affects cell metastasis. RON overexpression is observed in a variety of human cancers and exhibits increased expression with progression of the disease.

MET (also known as c-Met, cMet) is a receptor tyrosine kinase that is a heterodimeric protein comprising of a 50 kDa α-subunit and a 145 kDa β-subunit (Maggiora et al., J. Cell Physiol., 173:183-186, 1997). It is activated by binding to its natural ligand HGF (hepatocyte growth factor, also known as scatter factor) and signals via the PI3K and MAPK pathways. MET can be deregulated in cancer by mechanisms such as autocrine/paracrine HGF activation, over-expression of the receptor, and/or the presence of activating mutations. Significant expression of MET has been observed in a variety of human tumors, such as colon, lung, prostate (including bone metastases), gastric, renal, HCC, ovarian, breast, ESCC, and melanoma (Maulik et al., Cytokine & Growth Factor Reviews, 13:41-59, 2002). MET is also implicated in atherosclerosis and lung fibrosis. Inhibition of MET can cause a decrease in cell motility, proliferation and metastasis, as reviewed in, e.g., Chemical & Engineering News 2007, 85 (34), 15-23.

Elevated expression of MET has been detected in numerous cancers including lung, breast, colorectal, prostate, pancreatic, head and neck, gastric, hepatocellular, ovarian, renal, glioma, melanoma, and some sarcomas. See Christensen et al., Cancer Letters, 225(1):1-26 (2005); Comoglio et al., Nature Reviews Drug Disc., 7(6):504-516 (2008). MET gene amplification and resulting overexpression has been reported in gastric and colorectal cancer. Smolen et al., Proc. Natl. Acad. Sci. USA, 103(7):2316-2321 (2006); Zeng et al., Cancer Letters, 265(2):258-269 (2008). Taken together, the MET proto-oncogene has a role in human cancer and its over-expression correlates with poor prognosis. Abrogation of MET function with small molecule inhibitors, anti-MET antibodies or anti-HGF antibodies in preclinical xenograft model systems has shown impact when MET signaling serves as the main driver for proliferation and cell survival. Comoglio et al., Nature Reviews Drug Disc., 7(6):504-516 (2008); Comoglio et al., Cancer & Metastasis Reviews, 27(1):85-94 (2008).

As human cancers progress to a more invasive, metastatic state, multiple signaling programs regulating cell survival and migration programs are observed depending on cell and tissue contexts. Gupta et al., Cell, 127:679-695 (2006). Recent data highlight the transdifferentiation of epithelial cancer cells to a more mesenchymal-like state, a process resembling epithelial-mesenchymal transition (EMT) (Oft et al., Genes & Dev., 10:2462-2477 (1996); Perl et al., Nature, 392:190-193 (1998)) to facilitate cell invasion and metastasis. Brabletz et al., Nature Rev., 5:744-749 (2005); Christofori, Nature, 41:444-450 (2006). Through EMT-like transitions mesenchymal-like tumor cells are thought to gain migratory capacity at the expense of proliferative potential. A mesenchymal-epithelial transition (MET) has been postulated to regenerate a more proliferative state and allow macrometastases resembling the primary tumor to form at distant sites. Thiery, Nature Rev. Cancer, 2(6):442-454 (2002). MET and RON kinases have been shown to play a role in the EMT process. Camp et al., Cancer, 109(6):1030-1039 (2007); Grotegut et al., EMBO J., 25(15):3534-3545 (2006); Wang et al., Oncogene, 23(9):1668-1680 (2004). It has been documented in vitro that RON and MET can form heterodimers and signal via such RON-MET dimers.

MET and RON are known to interact and influence the activation of one another. Furthermore, co-expression of the two receptors, when compared to each receptor alone, is associated with the poorest clinical prognosis in bladder, CRC, and breast cancer patients. Since co-expression of RON and MET in cancer has been observed, such “cross-talk” may contribute to tumor growth.

ALK (Anaplastic Lymphoma Kinase) is a receptor tyrosine kinase that belongs to the insulin receptor subfamily. Constitutively active fusion proteins, activating mutations, or gene amplifications have been identified in various cancers, for example, kinase domain mutations in Neuroblastoma (Eng C., Nature, 2008, 455, 883-884), echinoderm microtubule-associated protein-like 4 (EML4) gene—ALK fusion in non-small cell lung cancer (NSCLC) (Soda M. et al., Nature, 2007, 448, 561-566), TPM3 and TPM4-ALK fusions in inflammatory myofibroblastic tumors (IMT) (Lawrence B. et al., Am. J. Pathol., 2000, 157, 377-384), and nucleophosmin (NPM)—ALK fusions in anaplastic large cell lymphomas (ALCL) (Morris S. W. et al., Science, 1994, 263, 1281-1284). Cell lines harboring such mutations or fusion proteins have been shown to be sensitive to ALK inhibition (McDermott U. et al., Cancer Res., 2008, 68, 3389-3395).

The following published documents are also noted: WO10/068,486; WO10/059,771; WO09/140,549; WO08/124849; WO08/051808; WO08/051805; WO08/039457; WO08/008,539; WO07/138472; WO07/132308; WO07/075567; WO07/067537; WO07/064,797; WO07/002433; WO07/002325; WO05/010005; WO05/004607; U.S. Pat. No. 7,452,993; U.S. Pat. No. 7,230,098; U.S. Pat. No. 6,235,769; US2009/005378; US2009/005356; US2008/293769; US2008/221148; US2008/167338; US2007/287711; US2007/123535; US2007/072874; US2007/066641; US2007/060633; US2007/049615; US2007/043068; US2007/032519; US2006/178374; US2006/128724; US2006/046991; US2005/182060; Wang et al., J. Appl. Poly. Sci., 109(5), 3369-3375 (2008); Zou et al., Cancer Res., 67(9), 4408 (2007); Arteaga, Nature Medicine, 13, 6, 675 (June 2007); Engelman, Science, 316, 1039 (May 2007) Saucier, PNAS, 101, 2345 (February 2004).

There is a need for effective active compounds and therapies for use in treating proliferative disease, including treatments for primary cancers, prevention of metastatic disease, and targeted therapies, including tyrosine kinase inhibitors, such as MET and/or RON inhibitors, IR, and IGF-1R inhibitors dual and multi-target inhibitors, including selective inhibitors (such as selectivity over Aurora kinase B (AKB) and/or KDR), and for potent, orally bioavailable, and efficacious inhibitors, and inhibitors that maintain sensitivity of epithelial cells to epithelial cell directed therapies.

### SUMMARY

In some aspects, the present invention concerns compounds of Formula I (and pharmaceutically acceptable salts thereof):

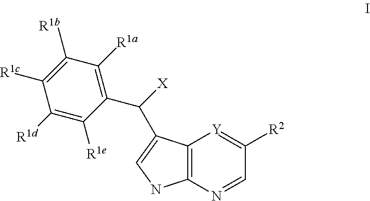

wherein X is haloaliphatic, Y is CH (which can be substituted) or N, R^{1a}—R^{1e} are independently optional substituents, and R² is an optional substituent. In some embodiments R² is optionally substituted heteroaryl.

The invention includes the Formula I compounds and salts thereof, their physical forms, preparation of the compounds, useful intermediates, and pharmaceutical compositions and formulations thereof.

In some aspects, compounds of the invention are useful as inhibitors of kinases, including in some aspects at least one of the MET, ALK, and RON kinases. In some aspects, compounds are active against IR and/or IGF-1R.

In some aspects, compounds of the invention are useful as inhibitors of kinases, including one or more of Trk, AXL, Tie-2, Flt3, FGFR3, Abl, Jak2, c-Src, IGF-1R, IR, PAK1, PAK2, and TAK1 kinases. In some aspects, compounds of the invention are inhibitors of kinases, including one or more of Blk, c-Raf, PRK2, Lck, Mek1, PDK-1, GSK3β, EGFR, p70S6K, BMX, SGK, CaMKII, and Tie-2 kinases.

In some aspects, compounds of the invention are useful as selective inhibitors of one or more of MET, RON, ALK, IR, or IGF-1R. In some embodiments, the compound is useful as a selective inhibitor of MET and/or RON and/or ALK over other kinase targets, such as KDR and/or Aurora kinase B (AKB). In some aspects, compounds of the invention are useful as selective inhibitors of MET, RON, ALK with selectivity over KDR and Aurora kinase B (AKB).

In some aspects, compounds of the invention are useful in treating proliferative disease, particularly cancers, including cancers, including cancers mediated or driven by one or more of MET, RON, ALK, IR, or IGF-1R, or other target(s), or cancers for which inhibition of such targets is useful alone or in combination with other active agents.

### DETAILED DESCRIPTION

### Compounds

In some aspects, the present invention concerns compounds and salts thereof of Formula I, above, wherein (Subgenus 1):

Y is CH or N;

X is C₁₋₃haloaliphatic;

R^{1a}, R^{1b}, R^{1c}, R^{1d}, and R^{1e} are each independently selected from H, halogen, —CN, C₁₋₆aliphatic, —OC₀₋₆aliphatic, —S(O)ₘC₁₋₆aliphatic, —SO₂N(C₀₋₆aliphatic)(C₀₋₆aliphatic), —N(C₀₋₆aliphatic)(C₀₋₆aliphatic), —N(C₀₋₆aliphatic)C(═O)C₀₋₆aliphatic, —N(C₀₋₆aliphatic)C(═O)OC₀₋₆aliphatic, —N(C₀₋₆aliphatic)C(═O)N(C₀₋₆aliphatic)(C₀₋₆aliphatic), —C(═O)C₀₋₆aliphatic, —C(═O)OC₀₋₆aliphatic, —C(═O)N(C₀₋₆aliphatic)(C₀₋₆aliphatic), —N(C₀₋₆aliphatic)-heterocyclyl, —N(C₀₋₆aliphatic)-heteroaryl, C₃₋₈cycloaliphatic, —O-cyclic, —O-heterocyclyl, sulfide, sulfoxide, or —S-cyclic, any of which is optionally substituted with one or more halogen, —CN, —OC₀₋₆aliphatic, —N(C₀₋₆aliphatic)(C₀₋₆aliphatic), —C(═O)N(C₀₋₆aliphatic)(C₀₋₆aliphatic), —C(═O)OC₀₋₆aliphatic, —C(═O)C₀₋₆aliphatic, heterocyclyl, or heteroaryl;

or heterocyclyl, which is optionally substituted with oxo, C₁₋₆aliphatic, C(═O)OC₁₋₆aliphatic, C(═O)C₀₋₆aliphatic, C(═O)N(C₀₋₆aliphatic)(C₀₋₆aliphatic), SO₂N(C₀₋₆aliphatic)(C₀₋₆aliphatic), SO₂(C₁₋₆aliphatic), heteroaryl, —S-heteroaryl, or —O-heteroaryl;

R² is selected from H, halo, —CN, —CF₃, —NO₂, C₀₋₆aliphatic, C₃₋₆cycloaliphaticC₀₋₆aliphatic, 3-6 membered heterocycloalkylC₀₋₆aliphatic, 3-6 membered heterocycloalkenylC₀₋₆aliphatic, arylC₀₋₆aliphatic, or heteroarylC₀₋₆aliphatic, any of which is optionally substituted with one or more G¹;
each G¹ is independently 4-10 membered heterocycloalkyl or heteroaryl optionally substituted with one or more OH, —CN, —OR⁶, R⁶, halogen, oxo, —NR⁶R⁷, —S(O)ₘR⁶, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —P(O)R^{a}R^{b}, —P(O)(R^{a})OR⁶, —P(O)(OR⁶)(OR⁷) or C₁₋₆alkyl, which is optionally substituted by halogen or —OC₀₋₅alkyl;

or G¹ is ₃₋₈cycloalkyl optionally substituted with one or more OH, —CN, —OR⁶, R⁶, halogen, oxo, —NR⁶R⁷, —S(O)ₘR⁶, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —P(O)R^{a}R^{b}, —P(O)(R^{a})OR⁶, —P(O)(OR⁶)(OR⁷) or —C₁₋₆alkyl which alkyl can be substituted by halogen or —OC₀₋₅alkyl;

or G¹ is C₁₋₆aliphatic optionally substituted with one or more —OH, —CN, —OR⁶, R⁶, halogen, oxo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, —P(O)R^{a}R^{b}, —P(O)(R^{a})OR⁶, —P(O)(OR⁶)(OR⁷), or 4-7 membered heterocycloalkyl optionally substituted by C₁₋₆alkyl;

wherein each R⁶, R⁷, R⁸, R⁹, R¹⁰, R^{a}, and R^{b} is independently C₀₋₅alkyl, C₃₋₆cycloalkyl, or 4-8 membered heterocycloalkyl optionally substituted with halogen, —OCF₃,

or —OC₀₋₃alkyl;

or —NR⁶R⁷ is 4-7 membered heterocycloalkyl optionally substituted with C₁₋₆alkyl;

or R⁸ and R⁹, R^{a} and R^{b}, R^{a} and OR⁶, or OR⁶ and OR⁷, taken together can combine with the atom that they are attached to form a 4-8 membered heterocycloalkyl or C₃₋₈cycloalkyl ring optionally substituted by C₁₋₆alkyl;

n is independently 0-7; and

m is independently 0-2.

In some aspects of Formula I or Subgenus 1 thereof (Subgenus 2):

Y is CH;

X is C₁₋₂haloalkyl; and

R² is selected from C₃₋₆cycloalkylC₀₋₆alkyl, 3-6 membered heterocycloalkylC₀₋₆alkyl, 3-6 membered heterocycloalkenylC₀₋₆alkyl, arylC₀₋₆alkyl, or heteroarylC₀₋₆alkyl, any of which is optionally substituted with 1-3 G¹.

In some aspects of Formula I or Subgenus 1-2 thereof (Subgenus 3):

Y is CH;

X is halomethyl; and

R² is a 5-membered heteroaryl which can be independently substituted with 1-2 G¹.

In some aspects of Formula I or Subgenus 1-3 thereof (Subgenus 4):

Y is CH; and

R² is

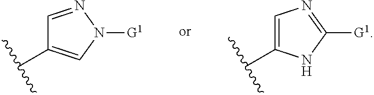

In some aspects of Formula I or Subgenus 1-4 thereof (Subgenus 5):

Y is CH;

R^{1a} and R^{1e} are each independently selected from halogen, —CN, C₁₋₃alkyl, —OC₀₋₃alkyl, wherein alkyl can be independently substituted with 1-3 fluorine atoms; and

R^{1b}, R^{1c}, and R^{1d} are each independently selected from H, halogen, —CN, C₁₋₃alkyl, —OC₀₋₃alkyl, wherein alkyl can be independently substituted with 1-3 fluorine atoms, —OC₀₋₆alkyl, —N(C₀₋₆alkyl)(C₀₋₆alkyl), —C(═O)N(C₀₋₆alkyl)(C₀₋₆alkyl), —C(═O)OC₀₋₆alkyl, —C(═O)C₀₋₆alkyl, or 5-6 membered heteroaryl.

In some aspects of Formula I or Subgenus 1-5 thereof (Subgenus 6):

Y is CH;

G¹ is C₁₋₆alkyl substituted with 0-3 substituents independently selected from OH, —CN, —OR⁶, —C(O)R^{b}, —C(O)NR⁶R⁷, —C(O)C(O)NR⁶R⁷, —C(O)OR⁶, —C(O)C(O)OR⁶, —OC(O)R^{b}, NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, (CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, —P(O)R^{a}R^{b}, —P(O)(R^{a})OR⁶, —P(O)(OR⁶)(OR⁷), or 4-7 membered heterocycloalkyl optionally substituted with C₁₋₆alkyl;

wherein each R⁶, R⁷, R⁸, R⁹, R¹⁰, R^{a}, and R^{b} are independently C₀₋₅alkyl or C₃₋₇cycloalkyl, each independently optionally substituted with halogen, —OCF₃, or —OC₀₋₃alkyl.

In some aspects of Formula I or Subgenus 1-5 thereof (Subgenus 7):

Y is CH;

G¹ is 4-8 membered heterocycloalkyl substituted with 0-3 substituents independently selected from OH, —CN, —OR⁶, halogen, R⁶, —S(O)ₘR⁶, —SO₂NR⁶R⁷, —C(O)R^{b}, —C(O)NR⁶R⁷, —C(O)C(O)NR⁶R⁷, —C(O)OR⁶, —C(O)C(O)OR⁶, —P(O)R^{a}R^{b}, —P(O)(R^{a})OR⁶, or —P(O)(OR⁶)(OR⁷);

or G¹ is C₃₋₈cycloalkyl substituted with 0-3 substituents independently selected from OH, —CN, —OR⁶, halogen, —S(O)ₘR⁶, —SO₂NR⁶R⁷, —C(O)R^{b}, —C(O)NR⁶R⁷, —C(O)C(O)NR⁶R⁷, —C(O)OR⁶, —C(O)C(O)OR⁶, —P(O)R^{a}R^{b}, —P(O)(R^{a})OR⁶, —P(O)(OR⁶)(OR⁷), or C₁₋₆alkyl optionally substituted with halogen or —OC₀₋₅alkyl;

wherein each R⁶, R⁷, R^{a}, and R^{b} is independently C₀₋₅alkyl or C₃₋₇cycloalkyl.

In some aspects of Formula I or Subgenus 1-7 thereof (Subgenus 8):

Y is CH;

R^{1b} and R^{1d} are each independently selected from H, halogen, —CN, C₁₋₃alkyl, or —OC₁₋₃alkyl, wherein alkyl can be substituted with 1-3 fluorine atoms; and

R^{1c} is H.

In some aspects of Formula I or Subgenus 1-5 and 7-8 thereof (Subgenus 9):

Y is CH; and

G¹ is C₃₋₈cycloalkyl substituted with 0-3 substituents independently selected from OH, —CN, —OR⁶, halogen, —S(O)ₘR⁶, —SO₂NR⁶R⁷, —C(O)R^{b}, —C(O)NR⁶R⁷, —C(O)OR⁶, —P(O)R^{a}R^{b}, —P(O)(R^{a})OR⁶, —P(O)(OR⁶)(OR⁷), or C₁₋₆alkyl optionally substituted with halogen or —OC₀₋₅alkyl;

wherein each R⁶, R⁷, R^{a}, and R^{b} is independently C₀₋₅alkyl or C₃₋₇cycloalkyl.

In some aspects of Formula I or Subgenus 1-5 and 7-8 thereof (Subgenus 10):

Y is CH; and

G¹ is 4-8 membered heterocycloalkyl substituted with 0-3 substituents independently selected from OH, —CN, —OR⁶, halogen, R⁶, —S(O)ₘR⁶, —SO₂NR⁶R⁷, —C(O)R^{b}, —C(O)NR⁶R⁷, —C(O)OR⁶, —P(O)R^{a}R^{b}, —P(O)(R^{a})OR⁶, or —P(O)(OR⁶)(OR⁷).

In some aspects of Formula I or Subgenus 1-10 thereof (Subgenus 11):

Y¹ is CH;

R^{1a} is halogen, or methoxy optionally substituted with 1-3 fluorine atoms; and

R^{1d} and R^{1e} are independently halogen.

In some aspects of Formula I or Subgenus 1-11 thereof (Subgenus 12):

Y is CH;

G¹ is 4-7 membered heterocycloalkyl optionally substituted with one or more independent halogen, —OH, —OCH₃, or C₁₋₃alkyl;

R^{1a} is halogen, or is methoxy optionally substituted with 1-3 fluorine atoms; and R^{1d} and R^{1e} are independently halogen.

In some aspects of Formula I or Subgenus 1-5, 7-9, and 11-12 thereof (Subgenus 13):

Y is CH;

G¹ is C₄₋₇cycloalkyl optionally substituted with one or more independent halogen, —OH, —OCH₃, or C₁₋₃alkyl;

R^{1a} is halogen, or is methoxy optionally substituted with 1-3 fluorine atoms; and

R^{1d} and R^{1e} are independently halogen.

In some aspects of Formula I or Subgenus 1-5, 7-9, and 11-13 thereof (Subgenus 14):

Y is CH;

G¹ is cyclohexanol;

R^{1a} is —OCHF₂;

R^{1d} is fluoro; and

R^{1e} is chloro.

In some aspects, the present invention concerns compounds and salts thereof of Formula I, which is present as a material that is a mixture of enantiomers.

In some aspects, the present invention concerns compounds and salts thereof of Formula I, which is present as a material that is substantially free of its (R)-1-(phenyl)fluoroethyl enantiomer.

In some aspects, the present invention concerns compounds and salts thereof of Formula I, which is present as a material that is substantially free of its (S)-1-(phenyl)fluoroethyl enantiomer.

In some aspects, the present invention concerns compounds and salts thereof of Formula I, which is present as a substantially pure material.

In some aspects, the present invention concerns compounds and salts thereof of Formula I, which exhibits inhibition of c-Met in a cellular mechanistic assay with an IC₅₀ of about 50 nM or less.

In some aspects, the present invention concerns compounds and salts thereof of Formula I, which exhibits inhibition of RON and/or ALK in a cellular mechanistic assay with an IC₅₀ of about 200 nM or less.

In some aspects, the present invention concerns compounds and salts thereof of Formula I, which is about 40-fold or more selective for c-Met over Aurora kinase B in cellular assays.

In some aspects, the present invention concerns compounds and salts thereof of Formula I selected from any one of Examples 1-137 herein.

In some aspects, the present invention concerns a pharmaceutical composition comprising the compound or salt according to Formula I, formulated with or without one or more pharmaceutical carriers.

In some aspects, the present invention concerns a method of treating a cancer mediated at least in part by RON and/or MET comprising administering to a mammal in need thereof a therapeutically effective amount of a compound or salt of Formula I.

In some aspects, the present invention concerns a method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of Formula I.

In some aspects, the present invention concerns a method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of Formula I, further comprising administering at least one additional anti-cancer agent in a therapeutically effective combination regimen.

In some aspects, the present invention concerns a method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of Formula I, further comprising administering at least one additional anti-cancer agent in a therapeutically effective combination regimen, wherein the agents in the combination regimen behave synergistically.

In some aspects, the present invention concerns a method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of Formula I, further comprising administering at least one additional anti-cancer agent in a therapeutically effective combination regimen, wherein the at least one additional anti-cancer agent comprises a VEGF, IGF-1R, or EGFR inhibitor.

In some aspects, the present invention concerns compounds and salts thereof of Formula I and their manufacture of a medicament for use in the method of treating a cancer selected from bladder, colorectal, non-small cell lung, breast, or pancreatic, ovarian, gastric, head and neck, prostate, hepatocellular, renal, glioma, or sarcoma cancer.

In some aspects, the present invention concerns compounds and salts thereof of Formula I and their manufacture of a medicament for use in the method of treating a cancer selected from bladder, colorectal, non-small cell lung, breast, or pancreatic, ovarian, gastric, head and neck, prostate, hepatocellular, renal, glioma, or sarcoma cancer, further comprising administering at least one additional anti-cancer agent in a therapeutically effective combination regimen.

The invention includes a compound of Formula I or a pharmaceutically acceptable salt thereof, which is sufficiently orally bioavailable for effective oral human administration.

The invention includes a compound of Formula I or a pharmaceutically acceptable salt thereof, which has a suitable therapeutic window for effective human administration, oral or otherwise.

The invention includes the compounds and salts thereof, and their physical forms, preparation of the compounds, useful intermediates, and pharmaceutical compositions and formulations thereof.

The compounds of the invention and term “compound” in the claims include any pharmaceutically acceptable salts or solvates, and any amorphous or crystal forms, or tautomers, whether or not specifically recited in context.

The invention includes the isomers of the compounds. Compounds may have one or more asymmetric carbon atoms can exist as two or more stereoisomers. Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. A single compound may exhibit more than one type of isomerism.

The present invention includes any stereoisomers, even if not specifically shown, individually as well as mixtures, geometric isomers, and pharmaceutically acceptable salts thereof. Where a compound or stereocenter is described or shown without definitive stereochemistry, it is to be taken to embrace all possible individual isomers, configurations, and mixtures thereof. Thus, a material sample containing a mixture of stereoisomers would be embraced by a recitation of either of the stereoisomers or a recitation without definitive stereochemistry. Also contemplated are any cis/trans isomers or tautomers of the compounds described.

Included within the scope of the invention are all stereoisomers, geometric isomers and tautomeric forms of the inventive compounds, including compounds exhibiting more than one type of isomerism, and mixtures of one or more thereof.

When a tautomer of the compound of Formula (I) exists, the compound of formula (I) of the present invention includes any possible tautomers and pharmaceutically acceptable salts thereof, and mixtures thereof, except where specifically stated otherwise.

The compounds of the invention are not limited to those containing all of their atoms in their natural isotopic abundance. The present invention includes compounds wherein one or more hydrogen, carbon or other atoms are replaced by different isotopes thereof. Such compounds can be useful as research and diagnostic tools in metabolism pharmacokinetic studies and in binding assays. A recitation of a compound or an atom within a compound includes isotopologs, i.e., species wherein an atom or compound varies only with respect to isotopic enrichment and/or in the position of isotopic enrichment. For nonlimiting example, in some cases it may be desirable to enrich one or more hydrogen atoms with deuterium (D) or to enrich carbon with ¹³C. Other examples of isotopes suitable for inclusion in the compounds of the invention include isotopes of hydrogen, chlorine, fluorine, iodine, nitrogen, oxygen, phosphorus, and sulfur. Certain isotopically-labeled compounds of the invention may be useful in drug and/or substrate tissue distribution studies. Substitution with heavier isotopes such as deuterium may afford certain therapeutic advantages resulting from greater metabolic stability, for example, increased in vivo half-life or reduced dosage requirements, and hence may be preferred in some circumstances. Substitution with positron emitting isotopes may be useful in Positron Emission Topography (PET) studies for examining substrate receptor occupancy.

Further, the compounds may be amorphous or may exist or be prepared in various crystal forms or polymorphs, including unsolvated, solvates and hydrates. The invention includes any such forms provided herein, at any purity level. A recitation of a compound per se means the compound regardless of any unspecified stereochemistry, physical form and whether or not associated with solvent or water.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g., D₂O, d6-acetone, d6-DMSO.

Also included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized.

The invention includes prodrugs of compounds of the invention which may, when administered to a patient, be converted into the inventive compounds, for example, by hydrolytic cleavage. Prodrugs in accordance with the invention can, for example, be produced by replacing appropriate functionalities present in the inventive compounds with certain moieties known to those skilled in the art as ‘pro-moieties’ as known in the art. Particularly favored derivatives and prodrugs of the invention are those that increase the bioavailability of the compounds when such compounds are administered to a patient, enhance delivery of the parent compound to a given biological compartment, increase solubility to allow administration by injection, alter metabolism or alter rate of excretion.

A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Compounds that are basic are capable of forming a wide variety of salts with various inorganic and organic acids. The acids that can be used to prepare pharmaceutically acceptable acid addition salts of such basic compounds are those that form acceptable acid addition salts. When the compound of the present invention is basic, its corresponding salt can be conveniently prepared from pharmaceutically acceptable acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Other salts are aspartate, besylate, bicarbonate/carbonate, bisulphate/sulfate, borate, camsylate, edisylate, gluceptate, glucuronate, hexafluorophosphate, hibenzate, hydrobromide/bromide, hydroiodide/iodide, malonate, methylsulfate, naphthylate, 2-napsylate, nicotinate, orotate, oxalate, palmitate, phosphate/hydrogen, phosphate/dihydrogen, phosphate, saccharate, stearate, tartrate, tosylate, and trifluoroacetate.

When the compound of the present invention is acidic, its corresponding salt can be conveniently prepared from pharmaceutically acceptable bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic bases include salts of primary, secondary, and tertiary amines, as well as cyclic amines and substituted amines such as naturally occurring and synthesized substituted amines. Other pharmaceutically acceptable organic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Other examples include benzathine, diolamine, glycine, meglumine, and olamine.

### Preparation

The invention includes the intermediates, examples, and synthetic methods described herein.

The compounds of the Formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art [such as those methods disclosed in standard reference books such as the Compendium of Organic Synthetic Methods, Vol. I-VI (Wiley-Interscience); or the Comprehensive Organic Transformations, by R. C. Larock (Wiley-Interscience)]. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed hereinbelow and the general skill in the art. Unless otherwise indicated, the substituents in the Schemes are defined as above. Isolation and purification of the products is accomplished by standard procedures, which are known to a chemist of ordinary skill.

When a general or exemplary synthetic procedure is referred to, one skilled in the art can readily determine the appropriate reagents, if not indicated, extrapolating from the general or exemplary procedures. Some of the general procedures are given as examples for preparing specific compounds. One skilled in the art can readily adapt such procedures to the synthesis of other compounds. Representation of an unsubstituted position in structures shown or referred to in the general procedures is for convenience and does not preclude substitution as described elsewhere herein. For specific groups that can be present, either as R groups in the general procedures or as optional substituents not shown, refer to the descriptions in the remainder of this document, including the claims, summary and detailed description.

### General Synthesis

Unless otherwise indicated, the substituents in the Schemes are defined as above. Isolation and purification of the products is accomplished by standard procedures, which are known to a chemist of ordinary skill. In the following general descriptions, R¹ indicates one or more substituents R^{1a}—R^{1e}.

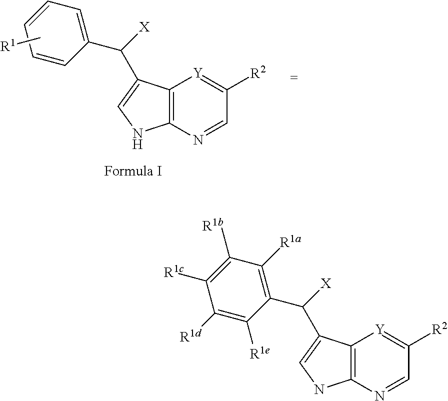

Compounds of Formula Ia {also known as 7-azaindoles or pyrrolo[2,3-b]pyridines} are compounds of Formula I wherein Y═CH. These compounds, or their pharmaceutically acceptable salts, can be prepared according to the reaction Schemes discussed hereinbelow and the general skill in the art.

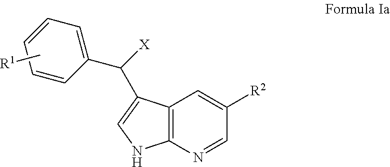

Compounds of Formula Ia wherein X═CH₂F can be prepared from compounds of Formula IIIa-A, or analogs of a compound of Formula IIIa-A wherein the hydroxyl group is replaced with an alkoxy group, as shown in Schemes 1-3 wherein R¹ and R² are as defined previously and A¹¹ is halogen such as Cl, Br, or I, or trifluoromethanesulfonate.

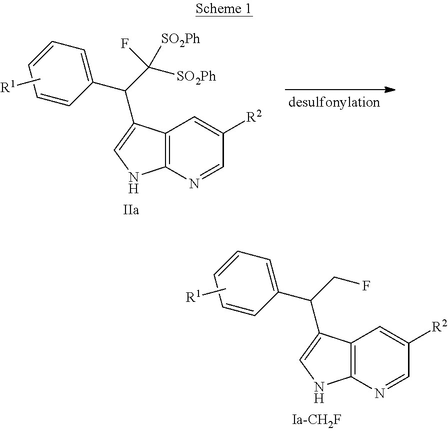

Compounds of Formula IIa can be desulfonylated to give compounds of Formula Ia-CH₂F (=Formula Ia wherein X═CH₂F) with reagents such as, but not limited to, sodium amalgam in buffered alcoholic solution or magnesium in methanol. The preferred reaction conditions for the desulfonylation with sodium amalgam will depend on the sodium content; for example, 20% sodium amalgam may allow the reaction to be conducted at −60 to −78° C. whereas 5% sodium amalgam may require higher temperatures, such as −20° C. to ambient temperature. Depending on the nature of substituents R¹ and R², the conditions may need to be modified to prevent formation of side products, such as, but not limited to, reduction of any halogen atoms present in R¹ or R². Suitable solvents for the desulfonylation include, but are not limited to, alcohols such as MeOH, EtOH, or isopropanol. Suitable buffer salts include, but are not limited to, disodium hydrogen phosphate, sodium dihydrogen phosphate, the corresponding potassium salts, or mixtures thereof.

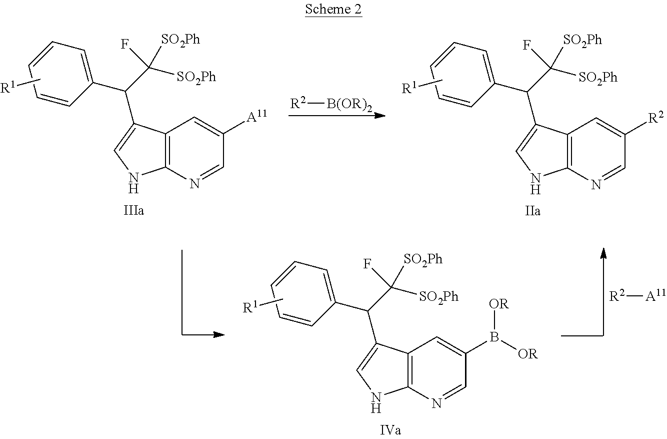

In a typical preparation of compounds of Formula IIa, a compound of Formula IIIa is reacted with a suitable boronic acid/ester [R²—B(OR)₂] in a suitable solvent via typical Suzuki coupling procedures. Suitable solvents for use in the above process include, but are not limited to, ethers such as THF, glyme, dioxane, dimethoxyethane, and the like; DMF; DMSO; MeCN; and alcohols such as MeOH, EtOH, isopropanol, trifluoroethanol, and the like. If desired, mixtures of these solvents can be used; however, preferred solvents are dimethoxyethane/water and dioxane/water. The above process can be carried out at temperatures between about 0° C. and about 120° C. Preferably, the reaction is carried out between 60° C. and about 100° C. The above process is preferably carried out at about atmospheric pressure although higher or lower pressures can be used. Substantially equimolar amounts of reactants are preferably used although higher or lower amounts can be used. One skilled in the art will appreciate that alternative methods may be applicable for preparing compounds of Formula IIa from IIIa. For example, compound of Formula IIIa could be reacted with a suitable organotin reagent R²—SnBu₃ or the like in a suitable solvent via typical Stille coupling procedures.

Alternatively, a compound of Formula IIIa may first be converted to a boronic acid or ester of formula IVa, followed by reaction with R²—A¹¹ via typical Suzuki coupling procedures as described above. In a typical preparation of a compound of formula IVa, a compound of Formula IIIa can be reacted with a suitable coupling partner [Bis(pinacolato)diboron or Pinacolborane)] in a suitable solvent under palladium catalysis. Suitable solvents for use in the above process include, but are not limited to, ethers such as THF, glyme, dioxane, dimethoxyethane, and the like; DMF; DMSO; MeCN; and alcohols such as MeOH, EtOH, isopropanol, trifluoroethanol, and the like. If desired, mixtures of these solvents can be used; however, preferred solvents are dioxane or DMSO. The above process can be carried out at temperatures between about 0° C. and about 120° C. Preferably, the reaction is carried out between 60° C. and about 100° C. The above process is preferably carried out at about atmospheric pressure although higher or lower pressures can be used. Substantially equimolar amounts of reactants used although higher or lower amounts can be used if desired. One skilled in the art will appreciate that alternative methods may be applicable for preparing compounds of Formula IVa, e.g., via halogen-metal exchange (for example, halogen-lithium exchange) and quench with borylation reagents such as tri-isopropyl borate. Furthermore, alternative methods may be applicable for preparing compounds of Formula IIa from R²—A¹¹, e.g., via typical Stille coupling procedures using the SnBu₃ analog of IVa.

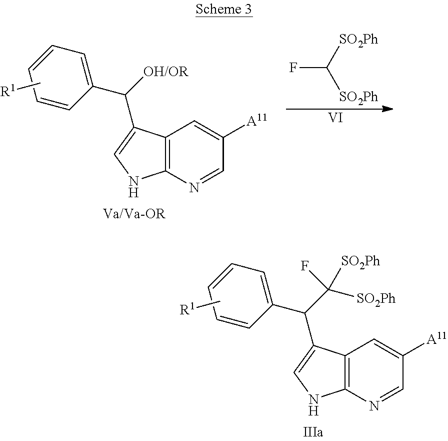

In a typical preparation of compounds of Formula IIIa, a compound of Formula Va or Va-OR is reacted first with thionyl chloride in a suitable solvent such as THF or chlorinated solvents like DCM or DCE, followed by evaporation to dryness. The residue is then redissolved in a solvent such as THF, and a solution of lithiated 1-(fluoro(phenylsulfonyl)methylsulfonyl)benzene (VI) is added at −78° C., followed by warming up to ambient temperature, to give IIIa.

Synthetic equivalents of a nucleophilic CH₂F group other than 1-(Fluoro(phenyl-sulfonyl)methylsulfonyl)benzene {also known as 1,1′-[(fluoromethanediyl)disulfonyl]dibenzene} are known in the literature and may be used here under similar conditions, e.g., 2-fluoro-1,3-benzodithiole-1,1,3,3-tetroxide (Angew. Chem. Int. Ed. 2010, 49, 1642-1647) and [(fluoromethyl)sulfonyl]benzene (J. Org. Chem. 2007, 72, 3119-3121).

Other haloalkyl groups X may be introduced in an analogous way to Schemes 1-3, as shown in Scheme 4.

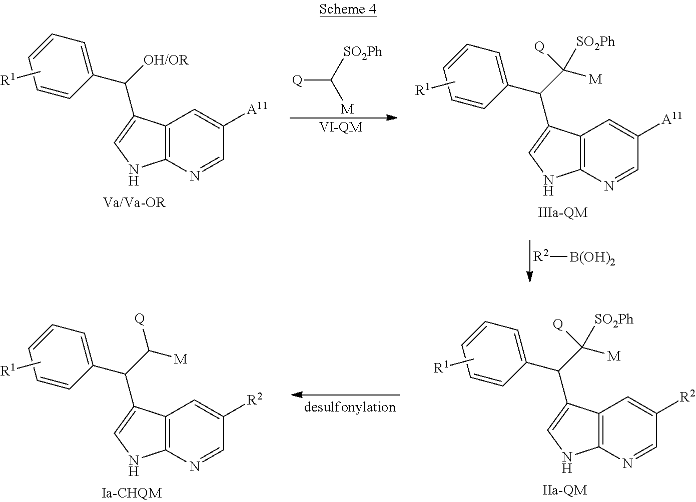

Compounds wherein X=higher 1-fluoroalkyl may be prepared following above scheme using VI-QM wherein Q=F and M=alkyl, e.g., CH₃ (reagent described in Chem. Pharm. Bull. 1996, 44, 703-708). Compounds wherein X═CHF₂ may be prepared following above scheme using VI-QM wherein Q=M=F (reagents described in J. Org. Chem. 2007, 72, 3119-3121 and J. Org. Chem. 2008, 73, 5699-5713). Compounds wherein X═CHCl₂ or CH₂Cl may be prepared following above scheme using VI-QM wherein Q=Cl and M=Cl or H, respectively (reagents described in J. Org. Chem. 2008, 73, 5699-5713).

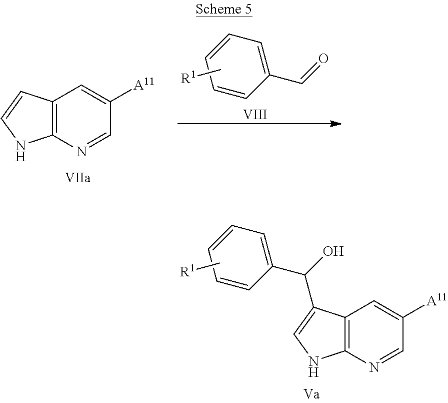

Compounds of Formula Va can be prepared as in Scheme 5, wherein R¹ is as defined previously and A¹¹ is halogen such as Cl, Br, or I. In a typical preparation, VIIa is treated with benzaldehyde VIII in a suitable solvent in the presence of a suitable base at a suitable reaction temperature. Suitable solvents for use in the above process include, but are not limited to, ethers such as THF, glyme, and the like; DMF, DMSO; MeCN; chlorinated solvents such as DCM or chloroform (CHCl₃); and alcohols such as MeOH, EtOH, isopropanol, or trifluoroethanol. If desired, mixtures of these solvents can be used or no solvent can be used. A preferred solvent is MeOH. Suitable bases for use in the above process include, but are not limited to, KOH, NaOH, LiOH, KOtBu, NaOtBu and NaHMDS and the like. A preferred base is KOH. The above process can be carried out at temperatures between about −78° C. and about 120° C. Preferably, the reaction is carried out between 20° C. and about 60° C. The above process to produce compounds of the present invention is preferably carried out at about atmospheric pressure although higher or lower pressures can be used. Substantially equimolar amounts of reactants are preferably used although higher or lower amounts can be used.

When alcohols are used as solvent, compounds of Formula Va-OR—analogs of compounds of Formula Va wherein the hydroxyl group is replaced with an alkoxy group—may also be obtained. For example, with MeOH as solvent one can obtain the methoxy analogs. Compounds of Formula Va and Va-OR can be interconverted: stirring Va in an alcohol ROH in the presence of an anhydrous acid (using, e.g., a solution of HCl in dioxane) converts it into Va-OR, while stirring Va-OR in aqueous acid (e.g., 2M aq, HCl) gives Va (Scheme 6).

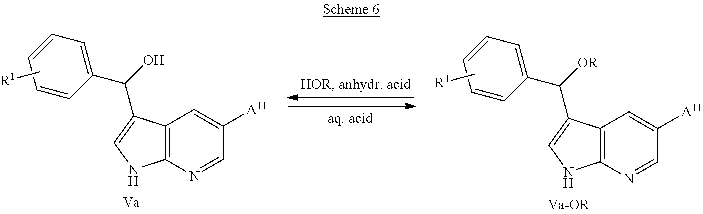

The benzaldehydes of formula VIII are commercially available or may be prepared by methods known to someone skilled in the art and the general literature such as the book Comprehensive Organic Transformations by R. C. Larock, or as described for the specific examples in this application. Various 7-azaindoles of formula VIIa are commercially available or may be prepared by methods known to someone skilled in the art and the general literature.

As will be apparent to the skilled artisan, the synthetic route/sequence can be modified as desired for the preparation of a given compound. For example, Group R² may be installed on compound VIIa under conditions similar to Scheme 2. The resulting compound can be treated with an appropriate benzaldehyde under conditions similar to Scheme 4, followed by introduction of a fluoromethyl group similar to Schemes 3 and 1.

### Scheme 7

R²—A¹¹→R²—B(OR)₂

The building block R²—B(OR)₂ may be prepared as in Scheme 7 from the building block R²—A¹¹, wherein R² is as defined previously, A¹¹ is halogen such as Cl, Br, or I, or trifluoromethanesulfonate, and B(OR)₂ is a suitable boronic acid/ester. The conversion may be accomplished by palladium catalysis under conditions similar to those described above in Scheme 2. An alternate route for compounds R²—A¹¹ wherein A¹¹ is Br or I consists of halogen-metal exchange with organolithium or -magnesium reagents followed by reaction with a boron reagent. Suitable reagents for A¹¹=I include, but are not limited to, iPrMgCl, iPrMgBr, or iPrMgCl.LiCl as organomagnesium reagents and MeOB(pinacol) or B(OMe)₃ as boron reagents. Suitable reagents for A¹¹=Br include, but are not limited to, nBuLi as organolithium reagent and MeOB(pinacol) or B(OMe)₃ as boron reagents.

The building blocks R²—A¹¹ and R²—B(OR)₂ wherein R²=substituted 4-pyrazolyl, 4(5)-imidazolyl, or 5-thiazolyl may be prepared as follows.

### R²=

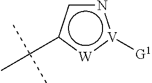

### R^{2a}=

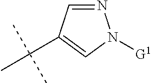

### R^{2b}=

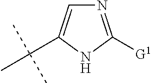

### R^{2c}=

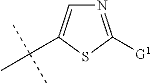

R^{2a}=R² wherein W—V═C—N; R^{2b}=R² wherein W—V═N—C; R^{2c}=R² wherein W—V═S—C.

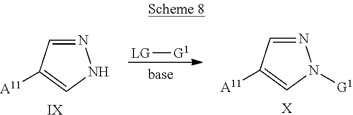

As shown in Scheme 8, building blocks containing R^{2a} may be prepared by alkylating a pyrazole IX that is unsubstituted on the nitrogen atoms with an alkylating agent LG-G¹, wherein LG is a leaving group such as the halogens Cl, Br, and I, or a sulfonate ester such as tosylate, mesylate, or trifluoromethanesulfonate. A¹¹ is halogen such as Cl, Br, or I. This reaction can also be conducted with pyrazoles that have a suitable boronic acid/ester B(OR)₂ in place of A¹¹.

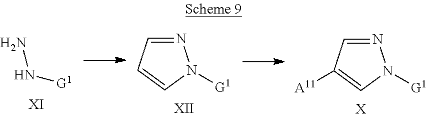

As shown in Scheme 9, the pyrazole ring in building blocks containing R^{2a} of Formula X may also be synthesized de novo by condensation of a hydrazine derivative H₂N—NH-G¹ with a malondialdehyde-type reagent (such as 1,1,3,3-tetramethoxypropane) followed by reaction with a halogenating agent to introduce A¹¹. Examples for halogenating agents include, but are not limited to, pyridinium perbromide or NBS (for A¹¹=Br), NIS or ICI (for A¹¹ ═I), or NCS (for A¹¹═Cl).

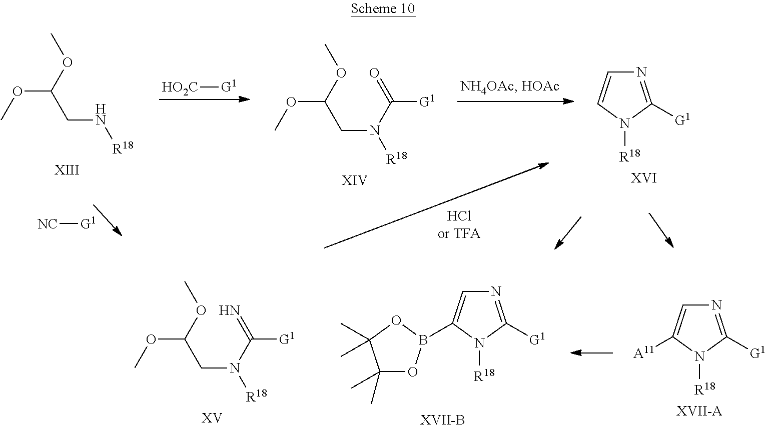

The imidazole ring in building blocks of Formula XVII-A/-B containing R^{2b}, wherein R¹⁸ is H, aliphatic, or cycloalkyl, may be synthesized de novo as shown in Scheme 10. The carboxylic acid HO₂C-G¹ is reacted with an aminoacetaldehyde acetal XIII under typical conditions for amide formation (e.g., EDCI+HOBt, mixed anhydrides, TBTU) to give an amide, which upon heating with NH₄OAc in acetic acid cyclizes to form the imidazole ring, yielding a compound of Formula XVI. R¹⁸ in the aminoacetaldehyde acetal XIII can be H, aliphatic, or cycloalkyl; if R¹⁸=H in XIII then it is convenient to introduce R¹⁸≠H by alkylation of XVI with R¹⁸—LG wherein LG is a leaving group such as Cl, Br, I, mesylate, tosylate, or triflate. In an alternate route to XVI, the aminoacetaldehyde acetal XIII can be reacted with the nitrile in the presence of CuCl without solvent to obtain the amidine of Formula XV, which is cyclized with HCl or TFA in alcoholic solvents such as methanol or ethanol to give the imidazole of Formula XVI (as described in Tetrahedron Letters 2005, 46, 8369-8372). The imidazole XVI can be halogenated at C5 to give a compound of Formula XVII-A with a suitable halogenating agent such as NBS (for A¹¹=Br), NIS or ICI (for A¹¹=I), or NCS (for A¹¹=Cl), in solvents such as THF, EtOAc, DCM, DMF, and the like. It can also be borylated at C5 to give a compound of Formula XVII-B with pinacolborane or bis(pinacolato)diboron in the presence of a catalyst consisting of an iridium complex and a 2,2′-bipyridine. Preferred catalysts include [Ir(OMe)(COD)]₂ and 2,2′-di-tert-butyl-bipyridine.

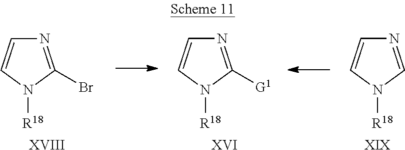

The imidazoles of Formula XVI may also be prepared from 2-bromoimidazoles XVIII or imidazoles XIX as shown in Scheme 11 by a variety of methods depending on the G¹ substituent. For example, the Br in XVIII may be displaced by nucleophiles or reacted in transition metal-catalyzed reactions. Bromine-lithium exchange generates an anion that can be reacted with electrophiles; the same anion can also be obtained by deprotonating XIX with a strong base such as LDA, LiTMP, or BuLi. Similar chemistry can be used for the corresponding thiazoles, starting from commercially available thiazole, 2-bromothiazole, or 2,5-dibromothiazole.

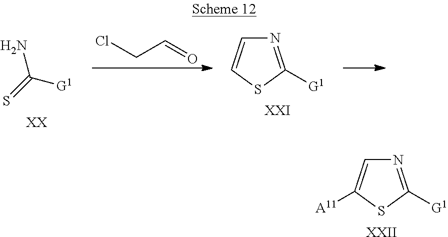

As shown in Scheme 12, the thiazole ring in building blocks containing R^{2c} of Formula XXII may also be synthesized de novo by condensation of a thioamide derivative H₂N—C(═S)-G¹ (XX) with chloroacetaldehyde—known to the skilled artisan as Hantzsch's synthesis—followed by reaction with a halogenating agent to introduce A¹¹.

Further methods of functionalizing and building up the pyrazole, imidazole, and thiazole rings can be found in the general literature, e.g., Volume 3 of Comprehensive Heterocyclic Chemistry II (Pergamon).

The functional groups present in R¹, R², X, and G¹ may be further modified by methods known to someone skilled in the art and the general literature such as the book Comprehensive Organic Transformations by R. C. Larock.

Compounds of Formula Ia have a chiral center at the carbon atom that connects the pyrrolo[2,3-b]pyridine core with X and the phenyl ring substituted with R¹. Enantiomerically pure compounds Ia can be prepared by various methods (Scheme 13).

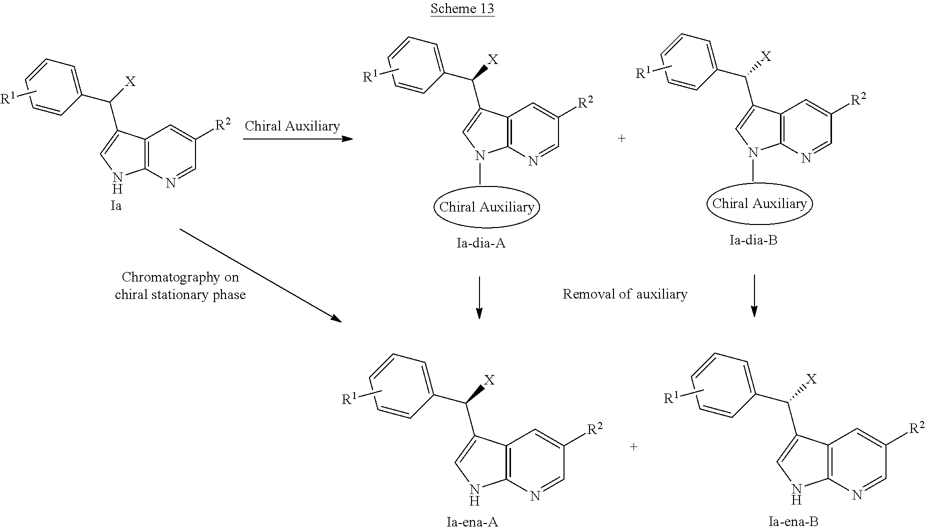

For example, enantiomerically pure Ia-ena-A and Ia-ena-B can be prepared by separation of racemic mixture Ia by chromatography on an enantiomerically pure stationary phase. Suitable chromatography systems for separation of racemic Ia include, but are not limited to, HPLC (high performance liquid chromatography) systems, SFC (supercritical fluid chromatography) systems and the like.

Alternatively, an enantiopure chiral auxiliary may be covalently attached to Ia to form the diastereomers Ia-dia-A and Ia-dia-B. After separation of these diastereomers by chromatography or crystallization, the chiral auxiliary is removed to reveal the separated enantiomers Ia-ena-A and Ia-ena-B. Suitable chiral auxiliaries for use in the above process include, but are not limited to, amino acids and their derivatives, (1S)-(+)-camphor-10-sulfonic acid, (1R)-(−)-camphor-10-sulfonic acid and the like.

One skilled in the art will appreciate that instead of covalently attaching a chiral auxiliary to compound Ia-A one may form diastereomeric salts that may be separated by crystallization. Neutralization of the separated diastereomeric salts provides the separated enantiomers of Ia. Suitable chiral acids or bases for salt formation include, but are not limited to amino acids and their derivatives, (1S)-(+)-camphor-10-sulfonic acid, (1R)-(−)-camphor-10-sulfonic acid and the like.

Instead of separating the racemic compounds of Formula Ia, it is also possible to separate at an earlier stage of the synthesis, for example, compounds of Formula IIa or IIIa by the same methods outlined above.

Compounds of Formula Ib {also known as pyrrolo[2,3-b]pyrazines} are compounds of Formula I wherein Y═N. These compounds, or their pharmaceutically acceptable salts, can be prepared according to the reaction Schemes 1-6 discussed for the compounds of Formula Ia and the general skill in the art.

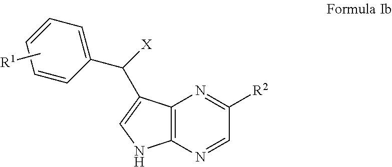

Compounds of Formula Ib have a chiral center at the carbon atom that connects the pyrrolopyrazine core with X and the phenyl ring substituted with R¹. Enantiomerically pure compounds Ib can be prepared by the methods discussed for the compounds of Formula Ia and the general skill in the art.

Racemic compounds of Formula Ia-CH₂F may be resolved into the enantiomers by any of the methods outlined above in schemes 6 and 7 and other methods known to someone skilled in the art.

As will be apparent to the skilled artisan, the synthetic routes/sequences can be modified as desired for the preparation of a given compound.

### Preparations and Intermediates

Unless otherwise noted, all materials/reagents were obtained from commercial suppliers and used without further purification. ¹H NMR (400 MHz or 300 MHz) and ¹³C NMR (100.6 or 75 MHz) spectra were recorded on Bruker or Varian instruments at ambient temperature with tetramethylsilane or the residual solvent peak as the internal standard. The line positions or multiples are given in ppm (δ) and the coupling constants (J) are given as absolute values in Hertz (Hz). The multiplicities in ¹H NMR spectra are abbreviated as follows: s (singlet), d (doublet), t (triplet), q (quartet), quint (quintet), m (multiplet), m_{c} (centered multiplet), br or broad (broadened), AA′BB′. The signal multiplicities in ¹³C NMR spectra were determined using the DEPT135 pulse sequence and are abbreviated as follows: +(CH or CH₃), −(CH₂), C_{quart} (C). Reactions were monitored by thin layer chromatography (TLC) on silica gel 60 F₂₅₄ (0.2 mm) precoated aluminum foil and visualized using UV light. Flash chromatography was performed with silica gel (400-230 mesh). Preparatory TLC was performed on Whatman LK6F Silica Gel 60 Å size 20×20 cm plates with a thickness of 500 or 1000 μm. Hydromatrix (=diatomaceous earth) was purchased from Varian. Mass-directed HPLC purification of compounds was performed on a Waters system composed of the following: 2767 Sample Manager, 2525 Binary Gradient Module, 600 Controller, 2996 Diode Array Detector, Micromass ZQ2000 for ionization, Phenomenex Luna 5μ C18(2) 100 Å 150×21.2 mm 5μ column with mobile phases of 0.01% Formic Acid Acetonitrile (A) and 0.01% Formic Acid in HPLC water (B), a flow rate of 20 mL/min, and a run time of 13 min. LC-MS data was collected on ZQ3 or TOF. ZQ3 is an Agilent 1100 HPLC equipped with a Series 1100 auto injector, a Series 1100 diode array detector, and Waters Micromass ZQ2000 for ionization. It uses the XBridge C18, 5μ particle size, 4.6×50 mm column with a mobile phase of Acetonitrile (A) and 0.01% Formic Acid in HPLC water (B). The flow rate is 1.0 mL/min, the run time is 5 min, and the gradient profiles are 0.00 min 5% A, 3.00 min 90% A, 3.50 min 90% A, 4.00 min 5% A, 5.00 min 5% A for polar_{—}5 min; 0.00 min 25% A, 3.00 min 99% A, 3.50 min 99% A, 4.00 min 25% A, 5.00 min 25% A for nonpolar_{—}5 min; and 0.00 min 40% A, 2.00 min 99% A, 3.00 min 99% A, 3.50 min 40% A, 5.00 min 40% A for vvnonpolar_{—}5 min. All Waters Micromass ZQ2000 instruments utilized electrospray ionization in positive (ES+) or negative (ES−) mode; it can also utilize atmospheric pressure chemical ionization in positive (AP+) or negative (AP−) mode. TOF is a Waters UPLC-LCT Premier system consisting of an ACQUITY UPLC equipped with an ACQUITY Sample Manager and LCT Premier XE MS for ionization. It uses an ACQUITY UPLC BEH®C18, 1.7 μm particle size, 2.1×50 mm column with a mobile phase of Acetonitrile (A) and 0.01% formic acid in water (B). The flow rate is 0.6 mL/min, run time is 3 min, and the gradient profile is 0.00 min 5% A, 0.2 min 5% A, 1.50 min 90% A, 2 min 90% A, 2.2 min 5% A, 3 min 5% A for polar_{—}3 min. The LCT Premier XE MS utilized electrospray ionization in positive (ES+) or negative (ES−), as well positive (AP+) or negative (AP−) in W mode. HPLC purification of compounds was performed on a Waters system consisting of a 2767 Sample Manager, 1525EF Binary Pump, and a 2487 Dual X Absorbance Detector. The system uses Phenomenex Luna C18(2), 5μ particle size, 50×21.2 mm columns with a mobile phase of Acetonitrile/0.25% Formic Acid and HPLC water/0.25% Formic Acid. The HPLC system for determination of enantiomeric purity consists of an Agilent 1100 HPLC and Chiralcel or Chiralpak 4.6×150 mm columns (Daicel Chemical Ind., Ltd.), eluting with acetonitrile/water mixtures. All melting points were determined with a Mel-Temp II apparatus and are uncorrected. Elemental analyses were obtained by Atlantic Microlab, Inc., Norcross, Ga.

### Intermediate 1: (5-Bromo-1H-pyrrolo[2,3-b]pyridin-3-yl)-(2,6-dichloro-3-fluorophenyl)methanol

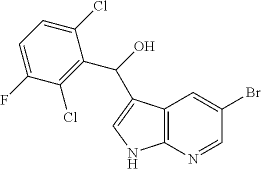 [SMILES: OC(c1c(Cl)ccc(F)c1Cl)c1c[nH]c2ncc(Br)cc12]

To a stirred mixture of 5-bromo-1H-pyrrolo[2,3-b]pyridine (0.100 g, 0.508 mmol) and 2,6-dichloro-3-fluorobenzaldehyde (0.107 g, 0.558 mmol) in MeOH (5 mL) was added potassium hydroxide (0.199 g, 3.55 mmol) at 0° C. under nitrogen atmosphere. The resulting mixture was then stirred at r.t. overnight. The mixture was then poured into water (50 mL), acidified with 2N HCl and extracted with ethyl acetate (50 mL×3). The organics were combined, dried (Na₂SO₄) and concentrated under reduced pressure to give a crude residue which was then purified by chromatography (eluent: 20% ethyl acetate in hexane). MS (ES+): m/z=388.85/390.84/392.83 [MH⁺]. HPLC: t_{R}=3.29 min (ZQ3, polar_{—}5 min).

### 2,6-Dichloro-3-fluorobenzaldehyde

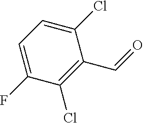 [SMILES: O=Cc1c(Cl)ccc(F)c1Cl]

To a solution of (2,6-Dichloro-3-fluorophenyl)methanol (100 g, 0.51 mol) in dichloromethane (450 mL) was added a solution of sodium bromide (54 g, 0.53 mol, in 90 mL water). The rapidly stirred biphasic mixture was cooled to −7° C. and TEMPO (1.54 g, 0.0100 mol) was added. A solution of 0.8 1M sodium hypochlorite (823 mL, 0.66 mol) saturated with sodium bicarbonate (75 g) was added dropwise over a period of 1 h while maintaining the temperature below −2° C. After the addition the reaction mixture was stirred for 30 min. The two layers separated and the DCM layer was washed with aq. solution of sodium thiosulfate. The DCM layer was dried (Na₂SO₄) and concentrated on rotary evaporator without using vacuum (aldehyde is volatile) to give the title compound as a solid, mp. 63-65° C. ¹H NMR (CDCl₃, 300 MHz): δ=7.23 (dd, 1H, J=7.8, 9.0 Hz), 7.35 (dd, 1H, J=4.5, 9.3 Hz), 10.2 (s, 1H).

Alternate Preparation:

To a solution of 2,4-dichloro-1-fluorobenzene (100 g, 0.606 mol) in THF (1.4 L) under nitrogen at −78° C., was added a 2.5 M solution of n-BuLi in hexanes (267 mL, 0.666 mol) dropwise over a period of 30 min, maintaining the temperature between −70 to −78° C. After 1.5 h stirring at −78° C., methyl formate (72.6 mL, 1.21 mol) was added slowly, and the reaction mixture was stirred overnight, warming up to rt. The reaction was quenched with sat. aqueous NH₄Cl (200 mL) and the organic layer was separated. The organic solvents were removed by distillation at atmosphere pressure and the crude material which contained a small amount of THF was crystallized from hexanes to give the title compound.

### (2,6-Dichloro-3-fluorophenyl)methanol

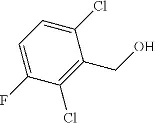 [SMILES: OCc1c(Cl)ccc(F)c1Cl]

To a solution of 2,6-Dichloro-3-fluorobenzoic acid (125 g, 0.59 mol) in THF (200 mL) was added BH₃.THF (592 mL, 592 mmol, 1 M solution in THF) dropwise at room temperature.

The reaction mixture was heated to reflux for 12 h. The borane was quenched with methanol (200 mL) and the resulting solution was concentrated to dryness. The residue was again co-evaporated with methanol to remove most of the trimethylborate. To the residue was added aq. sodium carbonate (50 g in 500 mL). The mixture was cooled and a white fine precipitate was filtered off to give the title compound. ¹H NMR (CDCl₃, 300 MHz): δ=2.10 (t, 1H, J=6.9 Hz), 4.96 (d, 2H, J=6.9 Hz), 7.09 (dd, 1H, J=8.1, 9.0 Hz), 7.29 (dd, 1H, J=4.8, 9.0 Hz).

### 2,6-Dichloro-3-fluorobenzoic acid

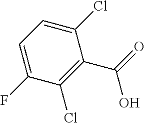 [SMILES: O=C(O)c1c(Cl)ccc(F)c1Cl]

To a cooled (−5° C.) solution of sodium hydroxide (252 g, 6.3 mol) in water (800 mL) was added bromine (86 mL, 1.68 mol) dropwise. The temperature of the reaction mixture was kept below −5° C. during the addition. A solution of 1-(2,6-Dichloro-3-fluorophenyl)ethanone (100 g, 480 mmol) in dioxane (800 ml) was added to the solution of sodium hypobromide in 1 h while maintaining the temperature below 0° C. The reaction mixture was warmed to room temperature and stirred for 2 h. After the TLC showed absence of starting material, the excess sodium hypobromide was destroyed with sodium sulfite (100 g in 100 mL water). The resulting solution was heated to 90° C. for 2 h. The reaction mixture was acidified with conc. HCl with vigorous stirring. The acidic solution was concentrated to remove all the dioxane and then extracted with dichloromethane (2×500 mL). The organic layer was dried (Na₂SO₄) and concentrated to give an oily residue, which after trituration with hexanes gave the title compound as a white solid. ¹H NMR (CDCl₃, 300 MHz): δ=7.20 (dd, 1H, J=8.7, 8.4 Hz), 7.33 (dd, 1H, J=9.3, 4.5 Hz).

### Intermediate 2: 5-Bromo-3-[(2-chloro-3-fluoro-6-methoxyphenyl)-hydroxymethyl]-1H-pyrrolo[2,3-b]pyridine

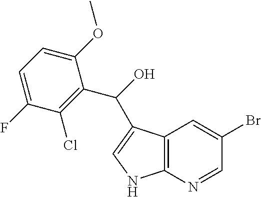 [SMILES: COc1ccc(F)c(Cl)c1C(O)c1c[nH]c2ncc(Br)cc12]

A solution of 2-chloro-3-fluoro-6-methoxybenzaldehyde (10.55 g, 55.82 mmol), 5-bromo-7-azaindole (10.0 g, 50.76 mmol) and KOH (4.0 g, 71 mmol) in methanol (200 mL) was stirred at ambient temperature for 12 h. The reaction mixture was quenched with water and the crystallizing solid was filtered and dried to give the title compound as a white solid. ¹H NMR (DMSO-d₆, 300 MHz):□δ=3.71 (s, 3H), 5.69 (d, 1H, J=6.3 Hz), 6.55 (d, 1H, J=4.5 Hz), 7.07 (dd, 1H, J=4.5, 4.2 Hz), 7.19 (s, 1H), 7.32 (t, J=8.0 Hz), 8.30 (s, 1H), 9.60 (s, 1H), 11.38 (brs, 1H).

### 2-Chloro-3-fluoro-6-methoxybenzaldehyde

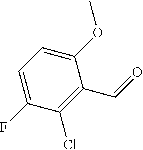

To a solution of 3-chloro-4-fluoroanisole (28.5 g, 178 mmol) in t-butyl methyl ether (200 mL, dried over anhydrous MgSO₄) at −78° C. was added 2.5 M n-butyl lithium in hexanes (107 mL, 267.5 mmol). After 3 h, methyl formate (18.76 mL) was added drop-wise while keeping the temperature below −60° C. The reaction mixture was quenched with sat. aq. ammonium chloride (250 mL) after 45 minutes and the organic layer was separated. The aq. layer was extracted with ethyl acetate (2×100 mL) and the combined organic layer was washed with water (200 mL) followed by brine, dried (Na₂SO₄) and concentrated to give a residue which on trituration with hexanes gave solids. The solids were filtered, taken again in hexanes and heated over steam bath. It was cooled, the light yellow desired product filtered and air dried to give the title compound. ¹H NMR (400 MHz, CDCl₃): δ=10.48 (d, J=0.8 Hz, 1H), 7.31 (dd, J=9.4, 7.8 Hz, 1H), 6.88 (dd, J=7.8, 3.8 Hz, 1H), 3.92 (s, 3H). ¹³C NMR (100.6 MHz, CDCl₃, DEPT135): δ=188.36 (+, J_{CF}=2.4 Hz), 158.01 (C_{quart}, J_{CF}=2.0 Hz), 152.73 (C_{quart}, J_{CF}=243.0 Hz), 122.87 (C_{quart}), 122.85 (C_{quart}, J_{CF}=18.4 Hz), 121.01 (+, J_{CF}=24.5 Hz), 110.65 (+, J_{CF}=6.9 Hz), 56.57 (+).

Alternative Preparation:

2-Chloro-3,6-difluorobenzaldehyde (10.0 g, 56.6 mmol) was dissolved in 50 mL of tetrahydrofuran and 120 mL of methanol. The reaction mixture was heated at 60° C. To the hot solution, a solution of sodium methoxide in methanol (25 weight %, 16 mL, 69 mmol) was added through an additional funnel over a period of 30 min. The reaction was heated at 60° C. for 16 hours. The reaction mixture was evaporated to remove the solvent on rotary evaporator, and water was added to the residue and stirred for 30 minutes. A solid separated out, which was filtered off and triturated with 10% ethyl acetate in hexanes to obtain the pure title compound (9.0 g, 85% yield).

### Intermediate 3: 5-Bromo-3-{[2-chloro-6-(difluoromethoxy)-3-fluorophenyl]-(methoxy)methyl}-1H-pyrrolo[2,3-b]pyridine

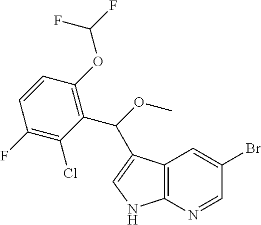 [SMILES: COC(c1c(OC(F)F)ccc(F)c1Cl)c1c[nH]c2ncc(Br)cc12]

To a solution of 5-bromo-7-azaindole (10.99 g, 55.80 mmol) in methanol (150 mL) was added 2-Chloro-6-difluoromethoxy-3-fluorobenzaldehyde (15.0 g, 66.7 mmol). A solution of KOH (4.69 g, 83.7 mmol) in 150 mL of methanol was added and stirred at room temperature for 48 h. The reaction mixture was poured into ice cold water and stirred for 30 min. A solid separated out, which was filtered off and dried in vacuo. ¹H NMR showed that it was a mixture of the title compound and (5-bromo-1H-pyrrolo[2,3-b]pyridin-3-yl)[2-chloro-6-(difluoromethoxy)-3-fluorophenyl]methanol in a ratio of =60:40. This mixture was converted to the pure title compound as follows:

To a solution of the (5-bromo-1H-pyrrolo[2,3-b]pyridin-3-yl)[2-chloro-6-(difluoromethoxy)-3-fluorophenyl]methanol/5-bromo-3-{[2-chloro-6-(difluoromethoxy)-3-fluorophenyl](methoxy)methyl}-1H-pyrrolo[2,3-b]pyridine mixture (23.0 g) in methanol (150 mL) was added 2 M HCl solution in diethyl ether (40.9 mL, 81.8 mmol), and the solution was stirred at room temperature for 16 h. Then the reaction mixture was poured into ice-cold sodium bicarbonate solution and stirred for 30 min. The precipitate was filtered off, washed with water and dried to yield the title compound (22.8 g). ¹H NMR (400 MHz, DMSO-d₆): δ=11.90 (s, 1H), 8.27 (d, J=2.0 Hz, 1H), 8.06 (d, J=2.0 Hz, 1H), 7.54 (dd, J=9.2, 8.8 Hz, 1H), 7.34 (dd, J=8.8, 4.8 Hz, 1H), 7.22 (s, 1H), 7.16 (dd, J=74.8, 72.4 Hz, 1H), 6.21 (s, 1H), 3.34 (s, 3H). ¹H NMR (400 MHz, CD₃OD): δ=8.22 (d, J=2.0 Hz, 1H), 8.07 (d, J=2.0 Hz, 1H), 7.33 (dd, J=8.8, 8.4 Hz, 1H), 7.26 (dd, J=9.2, 4.4 Hz, 1H), 7.22 (d, J=1.2 Hz, 1H), 6.73 (dd, J=76.0, 72.0 Hz, 1H), 6.32 (brs, 1H), 3.44 (s, 3H).

### 2-Chloro-6-difluoromethoxy-3-fluorobenzaldehyde

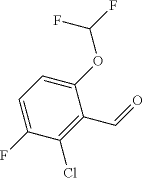 [SMILES: O=Cc1c(OC(F)F)ccc(F)c1Cl]

To 2-Chloro-4-difluoromethoxy-3-dimethoxymethyl-1-fluorobenzene (45.0 g, 166 mmol) was added acetic acid containing 20% water (80 ml) and heated at 50° C. for 16 h. The reaction mixture was cooled in an ice bath and basified with saturated aqueous sodium carbonate solution. The reaction mixture was extracted with ethyl acetate (200 mL, 100 ml); the combined organic layers were washed with brine, dried over sodium sulfate, filtered and concentrated to give crude product. It was purified by column chromatography on silica gel, eluting with 10% ethyl acetate in hexane. Pure compound isolated 28.0 g (75% yield). ¹H NMR (CDCl₃, 400 MHz): δ=10.41 (s, 1H), 7.37 (dd, J=8.8, 8.0 Hz, 1H), 7.22 (dd, J=9.2, 4.0 Hz, 1H), 6.58 (t, J=73.0 Hz, 1H).

Alternative Preparation:

To a solution of crude 2-chloro-4-difluoromethoxy-3-dimethoxymethyl-1-fluorobenzene (181 g, 670 mmol) in acetone (650 mL) and water (150 mL) was added Amberlyst-15 resin (540 g, pre-washed with water) and the mixture was stirred using mechanical stirrer for 40 h at RT. The Amberlyst-15 resin was removed by filtration using celite bed on sintered funnel, and the filtrate was evaporated on a rotary evaporator at RT (Note: aldehyde evaporates at higher temperatures under reduced pressure). The residue was purified by column chromatography on silica gel using ethyl acetate/hexanes (5% to 10%) to obtain the title compound (60 g, 40%).

### 2-Chloro-4-difluoromethoxy-3-dimethoxymethyl-1-fluorobenzene

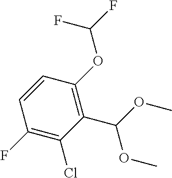 [SMILES: COC(OC)c1c(OC(F)F)ccc(F)c1Cl]

In a single neck flask, 3-chloro-2-dimethoxymethyl-4-fluorophenol (22 g, 100 mmol), sodium chlorodifluoroacetate (30.3 g, 200 mmol) and potassium carbonate (27.5 g, 200 mmol) were taken up in DMF (145 mL) under nitrogen atmosphere and heated at 90° C. for 16 h. The reaction mixture was cooled to room temperature, poured into water and extracted with ethyl acetate (2×200 mL, 100 mL). The combined organic layers were washed with water, dried over sodium sulfate, filtered and concentrated to give crude product, which was purified by column chromatography on silica gel using 10% ethyl acetate in hexane as an eluent to give 17 g (63% yield) of the title compound. ¹H NMR (CDCl₃, 300 MHz): δ=7.11-7.13 (m, 2H), 6.45 (t, J=75 Hz, 1H), 5.70 (s, 1H), 3.46 (s, 6H).

### 3-Chloro-2-dimethoxymethyl-4-fluorophenol

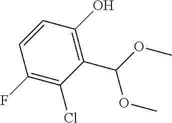

2-Chloro-3-fluoro-6-hydroxybenzaldehyde (79.0 g, 452 mmol) was taken in a single neck flask equipped with a condenser and a nitrogen inlet. To this, trimethylorthoformate (96.0 g, 99.0 mL, 905 mmol) and a solution of ammonium nitrate (3.6 g, 45 mmol) in methanol (40 mL) were added and heated to reflux for 16 hours. The reaction mixture was cooled to room temperature, poured into saturated aqueous sodium carbonate solution, stirred for few minutes, and extracted with ethyl acetate (300 mL, 200 mL). The combined organic layers were washed with water, dried over sodium sulfate, filtered and concentrated to give crude product. It was purified by column chromatography on silica gel using 10% ethyl acetate in hexane as eluent to give 65 g (64% yield) of the title compound. ¹HNMR (CDCl₃, 300 MHz): δ=8.52 (s, 1H), 7.04 (dd, J=9.0 Hz, 1H), 6.74-6.78 (m, 1H), 5.84 (s, 1H), 3.47 (s, 6H).

### 2-Chloro-3-fluoro-6-hydroxybenzaldehyde

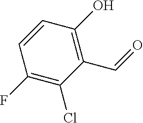 [SMILES: O=Cc1c(O)ccc(F)c1Cl]

2-Chloro-3-fluoro-6-methoxybenzaldehyde (46.0 g, 245 mmol) was added in a three neck flask equipped with a nitrogen inlet, a thermometer and an addition funnel. DCM (800 mL) was added and cooled to −70 to −78° C. using an acetone/dry ice bath. Boron tribromide (25.4 mL, 269 mmol) was diluted in 200 mL of dichloromethane and added to the reaction mixture slowly over a period of 1 h. The reaction mixture was allowed to warm to room temperature and stirred for 16 h. Then the reaction mixture was cooled to 0° C. in an ice bath and quenched by adding methanol (150 mL) over a period of 30 minutes and stirred at room temperature for 20 min. The solvents were removed, and the residue was diluted with dichloromethane and washed with aq. sodium bicarbonate solution followed by water. The organic layer was dried over sodium sulfate, filtered and concentrated to give crude product. It was purified by column chromatography on silica gel eluting with 2→3% methanol in dichloromethane, giving 34 g (80% yield) of the title compound. ¹HNMR (300 MHz, CDCl₃): δ=11.68 (s, 1H), 10.39 (s, 1H), 7.26-7.35 (m, 1H), 6.86-6.90 (m, 1H).

### Intermediate 4: 1-(trans-4-{[tert-butyl(dimethyl)silyl]oxy}cyclohexyl)-4-(4,4,5,5-tetramethyl-1,3,2-dioxaborolan-2-yl)-1H-pyrazole

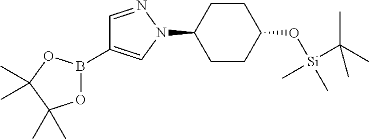 [SMILES: CC1(C)OB(c2cnn([C@H]3CC[C@H](O[Si](C)(C)C(C)(C)C)CC3)c2)OC1(C)C]

To a stirred solution of 1-(trans-4-{[tert-butyl(dimethyl)silyl]oxy}cyclohexyl)-4-iodo-1H-pyrazole (1.14 g, 2.80 mmol) in THF (30 mL) under nitrogen, cooled to 0° C., was added isopropylmagnesium chloride (2.0 M in THF, 2.3 mL, 4.6 mmol) dropwise over 5 minutes. The reaction mixture was stirred at 0° C. for 1 h, then 2-methoxy-4,4,5,5-tetramethyl-1,3,2-dioxaborolane (0.95 mL, 5.6 mmol) was added, and stirring was continued at RT for 1 h. Then sat. aq. NH4Cl solution (10 mL) was added, and the mixture was extracted with EtOAc (3×20 mL). The combined EtOAc extracts were washed with water (10 mL) and brine (15 mL), dried over Na2SO4, filtered, and concentrated under reduced pressure. The residue was purified by column chromatography on silica gel, eluting with 10% EtOAc in hexane to provide 1.10 g (96% yield) of the title compound. ¹H NMR (400 MHz, CD₃OD): δ=0.09 (s, 6H), 0.91 (s, 9H), 1.30 (s, 12H), 1.44-1.56 (m, 2H), 1.81-1.93 (m, 2H), 1.97-2.10 (m, 4H), 3.76 (tt, J=10.5, 4.3 Hz, 1H), 4.19 (tt, J=11.7, 3.9 Hz, 1H), 7.65 (s, 1H), 7.86 (s, 1H). MS (ES+): m/z=405.95/407.19/408.27 [MH⁺]. HPLC: t_{R}=3.21 min (ZQ3, polar_{—}5 min).

### 1-(trans-4-{[tert-Butyl(dimethyl)silyl]oxy}cyclohexyl)-4-iodo-1H-pyrazole

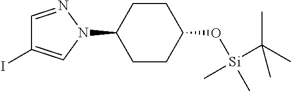 [SMILES: CC(C)(C)[Si](C)(C)O[C@H]1CC[C@H](n2cc(I)cn2)CC1]

A mixture of trans-4-(4-iodo-1H-pyrazol-1-yl)cyclohexanol (1.00 g, 3.42 mmol), tert-butyldimethylsilyl chloride (1.03 g, 6.85 mmol), 4-dimethylaminopyridine (80 mg, 0.7 mmol), imidazole (699 mg, 10.3 mmol) and DCM (20 mL, 300 mmol) was stirred rt for 20 min. The material was transferred to a separatory funnel, extracting with DCM and sat. NaHCO₃. The organic layer was dry-loaded onto silica gel for column chromatography, eluting with 3% EtOAc/hexanes. The fractions containing the pure product were concentrated in vacuo to afford the title compound as a clear oil that slowly solidified. Typical yields are ≧95%. ¹H NMR (400 MHz, DMSO-d₆): δ=0.05 (s, 6H), 0.86 (s, 9H), 1.33-1.47 (m, 2H), 1.70-1.91 (m, 4H), 1.96 (d, J=11.9 Hz, 2H), 3.58-3.75 (m, 1H), 4.11-4.21 (m, 1H), 7.49 (s, 1H), 7.92 (s, 1H). MS (ES+): m/z=407.05 (100) [MH⁺]. HPLC: t_{R}=3.22 min (vvnonpolar_{—}5 min, ZQ3).

### Trans- and cis-4-(4-Iodopyrazol-1-yl)cyclohexanol

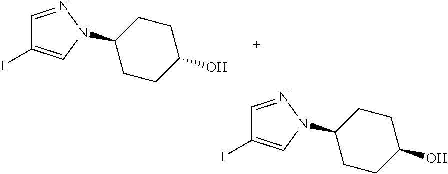 [SMILES: O[C@H]1CC[C@H](n2cc(I)cn2)CC1 ; O[C@H]1CC[C@@H](n2cc(I)cn2)CC1]

Sodium borohydride (0.29 g, 7.6 mmol) was added into the EtOH (20 mL) solution of 4-(4-iodopyrazol-1-yl)cyclohexanone (4.50 g, 15.5 mmol) at RT under an atmosphere of nitrogen. The mixture was stirred at RT for 2 h. Work-up: Solvent was evaporated and added water to the residue and extracted with EtOAc (3×60 mL). The combined organic extracts were dried over Na₂SO₄, filtered, and concentrated in vacuo to give an off-white solid. This material was purified by column chromatography on silica gel by eluting with 40% EtOAc/hexanes. The first (less polar) spot obtained was identified as cis isomer and the second (more polar) spot obtained was identified as trans isomer. Alternatively, the trans isomer may be isolated from the mixture of cis/trans isomers obtained in the reduction described above by crystallization from EtOAc/hexanes.

Cis-isomer: off-white solid, mp. 98-99° C. ¹H NMR (300 MHz, CDCl₃): δ=1.63-1.74 (m, 4H), 1.87-1.96 (m, 4H), 2.09-2.19 (m, 2H), 4.07-4.20 (m, 2H), 7.50 (s, 2H). ¹³C NMR (100.6 MHz, CDCl₃, DEPT135): δ=143.57 (+), 131.11 (+), 64.88 (+), 60.69 (+), 55.47 (C_{quart}), 31.59 (−), 27.09 (−).

Trans-isomer: white solid, mp. 82-86° C. ¹H NMR (400 MHz, CDCl₃): δ=1.42-1.51 (m, 2H), 1.79 (brs, 1H), 1.77-1.99 (m, 2H), 2.09-2.22 (m, 4H), 3.74 (br.tt, J=10.8, 4.0 Hz, 1H), 4.13 (tt, J=11.6, 3.8 Hz, 1H), 7.44 (d, J=0.4 Hz, 1H), 7.50 (d, J=0.4 Hz, 1H). ¹³C NMR (100.6 MHz, CDCl₃, DEPT135): δ=143.79 (+), 131.40 (+), 69.37 (+), 60.57 (+), 55.43 (C_{quart}), 33.93 (−), 30.94 (−). MS (ES+): m/z=293.11 [MH⁺]. HPLC: t_{R}=2.58 min (polar_{—}5 min, ZQ3).

### 4-(4-Iodopyrazol-1-yl)cyclohexanone

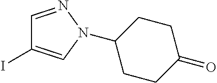 [SMILES: O=C1CCC(n2cc(I)cn2)CC1]

The mixture of 1-(1,4-dioxaspiro[4.5]dec-8-yl)-4-iodo-1H-pyrazole (20.0 g, 59.8 mmol), pyridinium p-toluenesulfonate (30.1 g, 120 mmol) in acetone (300 mL) and H₂O (300 mL) was heated at 65° C. for 16 h. The reaction mixture was partitioned between EtOAc (200 mL) and H₂O (100 mL), and the layers were separated. The aqueous layer was re-extracted with EtOAc (3×100 mL), and the combined organic fractions were washed with brine (1×), dried over Na₂SO₄, filtered and concentrated in vacuo resulting in 17.1 g (98% yield) of the title compound as a white solid. The material was used in the next step without further purification. ¹H NMR (400 MHz, CDCl₃): δ=7.54 (s, 1H), 7.52 (s, 1H), 4.62 (tt, J=4.0, 10.1 Hz, 1H), 2.64-2.38 (m, 6H), 2.36-2.24 (m, 2H). MS (ES+): m/z=291.00 [MH⁺]. HPLC: t_{R}=3.37 min (polar_{—}5 min, ZQ3).

### 1-(1,4-Dioxaspiro[4.5]dec-8-yl)-4-iodo-1H-pyrazole

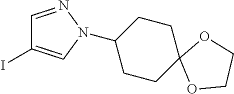 [SMILES: Ic1cnn(C2CCC3(CC2)OCCO3)c1]

A solution of 4-iodopyrazole (23.8 g, 123 mmol), 1,4-dioxaspiro[4.5]dec-8-yl 4-methylbenzenesulfonate (prepared according to U.S. Pat. No. 4,360,531) (42.2 g, 135 mmol), and Cs₂CO₃ (60.0 g, 184 mmol) in anhydrous degassed DMF (600 mL) was heated to 100° C. for 4 h. The reaction mixture was charged with an additional 1,4-dioxaspiro[4.5]dec-8-yl 4-methylbenzenesulfonate (5.20 g, 16.6 mmol) and Cs₂CO₃ (16.0 g, 49.1 mmol) and heated at 100° C. for an additional 16 h. The reaction mixture was cooled to ambient temperature, partitioned between EtOAc (400 mL) and sat. aq. NaHCO₃ solution (200 mL), and the layers were separated. The aqueous layer was re-extracted with EtOAc (3×150 mL), and the combined organic fractions were washed with H₂O (3×150 mL), brine (1×100 mL), dried over Na₂SO₄, filtered and concentrated in vacuo resulting in 45 g of an off-white solid. This solid was crystallized from i-PrOH (250 mL) and the white crystals were filtered through a fritted funnel resulting in the title compound as white crystals (31 g, 76% yield). A second crop of crystals from the mother liquor was slightly less pure. ¹H NMR (400 MHz, CDCl₃): δ=7.49 (s, 1H), 7.48 (s, 1H), 4.22 (tt, J=4.2, 11.2 Hz, 1H), 3.99-3.95 (m, 4H), 2.18-1.99 (m, 4H), 1.91-1.83 (m, 2H), 1.77-1.65 (m, 2H). MS (ES+): m/z=334.93 [MH⁺]. HPLC: t_{R}=3.74 min (polar_{—}5 min, ZQ3).

### EXAMPLES

### Example 1

### 3-[1-(2,6-Dichloro-3-fluorophenyl)-2-fluoroethyl]-5-(1-methyl-1H-pyrazol-4-yl)-1H-pyrrolo[2,3-b]pyridine

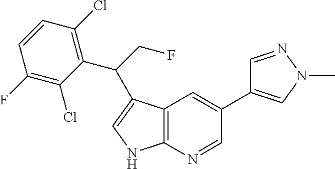 [SMILES: Cn1cc(-c2cnc3[nH]cc(C(CF)c4c(Cl)ccc(F)c4Cl)c3c2)cn1]

To a mixture of 3-[1-(2,6-dichloro-3-fluorophenyl)-2-fluoro-2,2-bis(phenylsulfonyl)ethyl]-5-(1-methyl-1H-pyrazol-4-yl)-1H-pyrrolo[2,3-b]pyridine (8.0 mg, 0.011 mmol) and disodium hydrogen phosphate (33.0 mg, 0.23 mmol) in MeOH (1.0 mL) and THF (0.30 mL) at −20° C. was added sodium amalgam (95:5 mercury: sodium, 0.098 g, 0.23 mmol). The resulting mixture was stirred at −10° C. for 2 h. The insoluble inorganic material was then removed by filtration. The remaining solution was diluted by MeOH (2.0 mL) and sat. aq. NH₄Cl solution (2.0 mL). The solvent was removed under reduced pressure to give a crude residue which was purified by silica gel column (30% EtOAc in DCM) to give the title compound. ¹H NMR (400 MHz, CD₃OD): δ=3.95 (s, 3H), 5.37 (ddd, J=39.7, 8.8, 6.8 Hz, 2H), 5.61-5.74 (m, 1 H), 7.29 (t, J=8.6 Hz, 1H), 7.36 (s, 1H), 7.44-7.57 (m, 1H), 7.61-7.67 (m, 2H), 7.86 (s, 1 H), 8.38 (d, J=1.8 Hz, 1H). MS (ES+): m/z=407.02/409.02/411.02 [MH⁺]. HPLC: t_{R}=1.38 min (polar_{—}3 min, TOF).

### 3-[1-(2,6-Dichloro-3-fluorophenyl)-2-fluoro-2,2-bis(phenylsulfonyl)ethyl]-5-(1-methyl-1H-pyrazol-4-yl)-1H-pyrrolo[2,3-b]pyridine

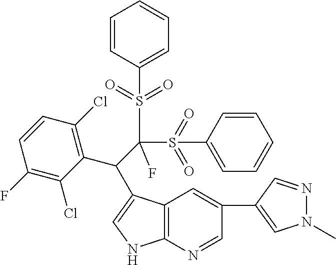 [SMILES: Cn1cc(-c2cnc3[nH]cc(C(c4c(Cl)ccc(F)c4Cl)C(F)(S(=O)(=O)c4ccccc4)S(=O)(=O)c4ccccc4)c3c2)cn1]

To a stirred mixture of 1-methyl-4-(4,4,5,5-tetramethyl-1,3,2-dioxaborolan-2-yl)-1H-pyrazole (9.0 mg, 0.043 mmol), 5-bromo-3-[1-(2,6-dichloro-3-fluorophenyl)-2-fluoro-2,2-bis(phenylsulfonyl)ethyl]-1H-pyrrolo[2,3-b]pyridine (20.0 mg, 0.029 mmol) and potassium fluoride (5.0 mg, 0.087 mmol) in 1,4-dioxane (4.0 mL) and H₂O (1.0 mL) was added (1,1′-bis-(diphenylphosphino)-ferrocene)palladium dichloride (2.1 mg, 0.0029 mmol) under Nitrogen. The resulting mixture was stirred at 90° C. for 1 h. The solvent was removed under reduced pressure to give a crude residue which was purified by silica gel column (20% EtOAc in DCM) to give the title compound. MS (ES+): m/z=687.05/689.05/692.04 [MH⁺]. HPLC: t_{R}=1.45 min (polar_{—}3 min, TOF).

### 5-Bromo-3-[1-(2,6-dichloro-3-fluorophenyl)-2-fluoro-2,2-bis(phenylsulfonyl)ethyl]-1H-pyrrolo[2,3-b]pyridine

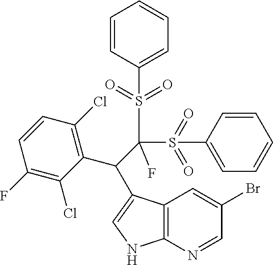 [SMILES: O=S(=O)(c1ccccc1)C(F)(C(c1c(Cl)ccc(F)c1Cl)c1c[nH]c2ncc(Br)cc12)S(=O)(=O)c1ccccc1]

To a stirred solution of 1,1′-[(fluoromethanediyl)disulfonyl]dibenzene [prepared as described in J. Org. Chem. 2008, 73 (15), 5699-5713](978 mg, 3.1 mmol) in THF (8.0 mL) was added 2.5 M of n-BuLi in hexane (1.45 mL, 3.63 mmol) at −78° C., the resulting mixture was stirred for 30 min at −78° C. before use. To a stirred solution of (5-bromo-1H-pyrrolo[2,3-b]pyridin-3-yl)(2,6-dichloro-3-fluorophenyl)methanol (Intermediate 1) (402.0 mg, 1.03 mmol) in anhydrous THF (5.0 mL) was added thionyl chloride (0.22 mL, 3.11 mmol) at 0° C. The resulting mixture was stirred for 30 min at rt, then the solvent was removed under nitrogen and the residue was dried under high vacuum. This residue was dissolved in anhydrous THF (15.0 mL) and cooled to −78° C.; to this solution was then added the previously prepared mixture described above {2.5 M of n-BuLi and 1,1′-[(fluoromethanediyl)disulfonyl]dibenzene in THF at −78° C.} by cannula at −78° C. The resulting mixture was allowed to warm up to rt in about 1 hour. The reaction was quenched by adding sat. aq. NH₄Cl solution (5.0 mL). The bulk of solvent was removed under reduced pressure to give a residue, which was diluted by DCM (20.0 mL) and extracted by DCM (20.0 mL×3). The organic phases were combined, dried (Na₂SO₄) and concentrated in vacuo to give a crude residue that was purified by silica gel chromatography (eluent: 10% EtOAc in DCM) to give the title compound. ¹H NMR (400 MHz, CD₃OD): δ=6.64 (dd, J=38.7, 5.6 Hz, 1H), 7.16-7.22 (m, 3H), 7.25 (t, J=7.8 Hz, 2 H), 7.38-7.45 (m, 1H), 7.49-7.54 (m, 1H), 7.54-7.70 (m, 3H), 7.76-7.83 (m, 1H), 7.93-8.05 (m, 3H), 8.20-8.24 (m, 1H). MS (ES+): m/z=684.90/686.90/688.90 [MH⁺]. HPLC: t_{R}=1.66 min (polar_{—}3 min, TOF).

### Intermediate 5: trans-4-(4-{3-[-1-(2-Chloro-3-fluoro-6-methoxyphenyl)-2-fluoroethyl]-1H-pyrrolo[2,3-b]pyridin-5-yl}-1H-pyrazol-1-yl)cyclohexanol

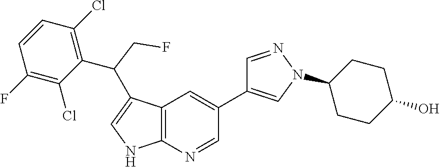 [SMILES: O[C@H]1CC[C@H](n2cc(-c3cnc4[nH]cc(C(CF)c5c(Cl)ccc(F)c5Cl)c4c3)cn2)CC1]

To a mixture of 5-[1-(trans-4-{[tert-butyl(dimethyl)silyl]oxy}cyclohexyl)-1H-pyrazol-4-yl]-3-[1-(2-chloro-3-fluoro-6-methoxyphenyl)-2-fluoro-2,2-bis(phenylsulfonyl)ethyl]-1H-pyrrolo[2,3-b]pyridine (470.0 mg, 0.53 mmol) and disodium hydrogen phosphate (1.5 g, 10.6 mmol) in MeOH (15.0 mL) at −20° C. was added sodium amalgam (95:5 mercury: sodium, 4.5 g, 10.6 mmol). The resulting mixture was stirred between −15° C. and −5° C. for 1.5 h. The mixture was transferred into another flask by filtration to remove inorganic insoluble material. Sat. aq. solution of NH₄Cl (2 mL) was added to the mixture, then the solvent was removed under reduced pressure to give a residue, which was diluted by DCM (10.0 mL) and extracted by DCM (20.0 mL×3). The combined organic phases were dried (Na₂SO₄) and concentrated to give a crude product (TBDMS ether of the title compound), which was used for the next step immediately without any further purifications. MS (ES+): m/z=601.25/603.25 [MH⁺]. HPLC: t_{R}=1.96 min (polar_{—}3 min, TOF).

The crude material prepared above was dissolved in THF (10.00 mL) at 0° C., 4.0 M of HCl in H₂O (4.0 mL, 16.0 mmol) was added at 0° C., and the resulting mixture was stirred at rt for 30 min. NaHCO₃ (1.56 g, 18.6 mmol) was added slowly to the mixture. Then the solvent was removed under reduced pressure to give a residue, which was diluted by DCM (10 mL) and extracted by DCM (20 mL×3). The combined organic phases were dried (Na₂SO₄) and concentrated to give a crude residue that was purified by silica gel chromatography (eluent: 5% MeOH in DCM) to give the title compound (70% yield over 2 steps). ¹H NMR (400 MHz, CD₃OD): δ=1.46-1.60 (m, 2H), 1.89-2.04 (m, 2H), 2.08-2.24 (m, 4H), 3.71 (tt, J=11.0, 4.2 Hz, 1H), 3.82 (s, 3H), 4.24 (tt, J=11.8, 3.8 Hz, 1H), 5.15 (ddd, J=31.1, 9.4, 8.1 Hz, 1H), 5.28 (ddd, J=30.8, 8.6, 7.3 Hz, 1H), 5.38-5.49 (m, 1H), 7.01 (dd, J=9.1, 4.3 Hz, 1H), 7.20 (t, J=8.8 Hz, 1H), 7.30 (s, 1H), 7.75 (d, J=0.5 Hz, 1H), 7.96 (d, J=1.8 Hz, 1H), 8.01 (s, 1H), 8.38 (d, J=2.0 Hz, 1H). MS (ES+): m/z=487.11/489.12 [MH⁺]. HPLC: t_{R}=1.29 min (polar_{—}3 min, TOF).

### 5-[1-(trans-4-{[tert-Butyl(dimethyl)silyl]oxy}cyclohexyl)-1H-pyrazol-4-yl]-3-[1-(2-chloro-3-fluoro-6-methoxyphenyl)-2-fluoro-2,2-bis(phenylsulfonyl)ethyl]-1H-pyrrolo[2,3-b]pyridine

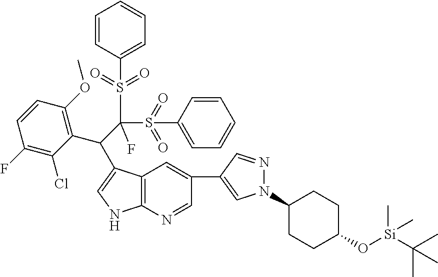 [SMILES: COc1ccc(F)c(Cl)c1C(c1c[nH]c2ncc(-c3cnn([C@H]4CC[C@H](O[Si](C)(C)C(C)(C)C)CC4)c3)cc12)C(F)(S(=O)(=O)c1ccccc1)S(=O)(=O)c1ccccc1]

To a stirred mixture of 1-(trans-4-{[tert-butyl(dimethyl)silyl]oxy}cyclohexyl)-4-(4,4,5,5-tetramethyl-1,3,2-dioxaborolan-2-yl)-1H-pyrazole (Intermediate 4) (298.0 mg, 0.73 mmol), 5-bromo-3-[1-(2-chloro-3-fluoro-6-methoxyphenyl)-2-fluoro-2,2-bis(phenylsulfonyl)ethyl]-1H-pyrrolo[2,3-b]pyridine (400.0 mg, 0.58 mmol) and potassium fluoride (102.2 mg, 1.75 mmol) in 1,4-dioxane (10.0 mL) and H₂O (2.5 mL) was added (1,1′-bis-(diphenylphosphino)-ferrocene) palladium dichloride (21.4 mg, 0.029 mmol) under nitrogen atmosphere. The resulting mixture was then stirred at 90° C. for 90 min. The solvent was removed under reduced pressure to give a residue, which was purified by silica gel chromatography (eluent: 20→30% EtOAc in DCM) to give the title compound (82% yield). ¹H NMR (400 MHz, CD₃OD): δ=0.13-0.15 (m, 6H), 0.94-0.97 (m, 9H), 1.51-1.65 (m, 2H), 1.93-2.25 (m, 6H), 3.67 (s, 3H), 3.79-3.88 (m, 1H), 4.26-4.32 (m, 1H), 6.44-6.59 (m, 2H), 7.03 (t, J=9.1 Hz, 1H), 7.12-7.19 (m, 2H), 7.22-7.29 (m, 2H), 7.31-7.39 (m, 2H), 7.50-7.57 (m, 2H), 7.68-7.74 (m, 1H), 7.79-7.88 (m, 3H), 8.08 (d, J=0.5 Hz, 1H), 8.14 (d, J=2.0 Hz, 1H), 8.37 (d, J=2.0 Hz, 1H). MS (ES+): m/z=881.24/883.24 [MH⁺]. HPLC: t_{R}=1.94 min (polar_{—}3 min, TOF).

### 5-Bromo-3-[1-(2-chloro-3-fluoro-6-methoxyphenyl)-2-fluoro-2,2-bis(phenylsulfonyl)ethyl]-1H-pyrrolo[2,3-b]pyridine

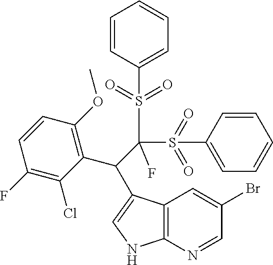 [SMILES: COc1ccc(F)c(Cl)c1C(c1c[nH]c2ncc(Br)cc12)C(F)(S(=O)(=O)c1ccccc1)S(=O)(=O)c1ccccc1]

To a stirred solution of 1,1′-[(fluoromethanediyl)disulfonyl]dibenzene (978.2 mg, 3.11 mmol) in THF (8.0 mL) was added 2.5 M of n-BuLi in hexane (1.45 mL, 3.63 mmol) at −78° C.; the resulting mixture was stirred for 30 min at −78° C. before use. To a stirred solution of (5-bromo-1H-pyrrolo[2,3-b]pyridin-3-yl)(2-chloro-3-fluoro-6-methoxyphenyl)methanol (Intermediate 2) (400.0 mg, 1.03 mmol) in anhydrous THF (5.00 mL) was added thionyl chloride (0.22 mL, 3.11 mmol) at 0° C. The resulting mixture was stirred for 30 min at rt, then the solvent was removed under nitrogen and the residue was dried under high vacuum. This residue was dissolved in anhydrous THF (15.00 mL) and cooled to −78° C.; to this solution was then added the previously prepare mixture described above {2.5 M of n-BuLi and 1,1′-[(fluoromethanediyl)disulfonyl]dibenzene in THF at −78° C.} by cannula at −78° C. The resulting mixture was allowed to warm up to rt in about 1 hour. The reaction was quenched by sat. aq. NH₄Cl solution (5.0 mL). The bulk of solvent was removed under reduced pressure to give a residue, which was diluted by DCM (20.0 mL) and extracted by DCM (20.0 mL×3). The organic phase were combined, dried (Na₂SO₄) and concentrated to give a crude residue which was purified by silica gel chromatography (eluent: 10% EtOAc in DCM) to give the title compound (85% yield). MS (ES+): m/z=680.97/682.97/684.97 [MH⁺]. HPLC: t_{R}=1.61 min (polar_{—}3 min, TOF).

### Examples 2 & 3

### trans-4-(4-{3-[(1R)-1-(2-chloro-3-fluoro-6-methoxyphenyl)-2-fluoroethyl]-1H-pyrrolo[2,3-b]pyridin-5-yl}-1H-pyrazol-1-yl)cyclohexanol and trans-4-(4-{3-[(1S)-1-(2-chloro-3-fluoro-6-methoxyphenyl)-2-fluoroethyl]-1H-pyrrolo[2,3-b]pyridin-5-yl}-1H-pyrazol-1-yl)cyclohexanol

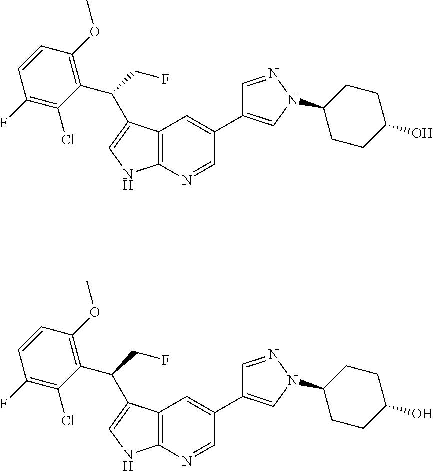 [SMILES: COc1ccc(F)c(Cl)c1[C@H](CF)c1c[nH]c2ncc(-c3cnn([C@H]4CC[C@H](O)CC4)c3)cc12 ; COc1ccc(F)c(Cl)c1[C@@H](CF)c1c[nH]c2ncc(-c3cnn([C@H]4CC[C@H](O)CC4)c3)cc12]

The racemic compound of Intermediate 5 was subjected to SFC separation on a chiral stationary phase to give two enantiomers. Preparative SFC (ChiralPak IA 21×250 mm I.D., solvent 60:40 scCO₂/isopropanol (0.2% isopropylamine) isocratic, flow rate 30 mL/min, UV detection at 254 nm): t_{R}=10.32 min [(1R) enantiomer=Example 2]; t_{R}=14.72 min [(1S) enantiomer=Example 3]. ¹HNMR and LC-MS data for both enantiomers are identical to the data obtained from the racemic mixture.

### Intermediate 6: trans-4-[4-(3-{-1-[2-Chloro-6-(difluoromethoxy)-3-fluorophenyl]-2-fluoroethyl}-1H-pyrrolo[2,3-b]pyridin-5-yl)-1H-pyrazol-1-yl]cyclohexanol

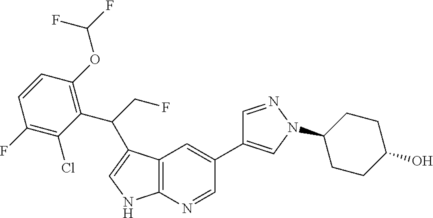 [SMILES: O[C@H]1CC[C@H](n2cc(-c3cnc4[nH]cc(C(CF)c5c(OC(F)F)ccc(F)c5Cl)c4c3)cn2)CC1]

To a mixture of 5-[1-(trans-4-{[tert-butyl(dimethyl)silyl]oxy}cyclohexyl)-1H-pyrazol-4-yl]-3-{1-[2-chloro-6-(difluoromethoxy)-3-fluorophenyl]-2-fluoro-2,2-bis(phenylsulfonyl)ethyl}-1H-pyrrolo[2,3-b]pyridine (2.0 g, 2.18 mmol) and disodium hydrogen phosphate (3.7 g, 26.16 mmol, 12 eq.) in MeOH/THF (anhydrous MeOH: 50.0 mL, anhydrous THF: 10.0 mL) at −78° C. was added sodium amalgam (80:20 mercury: sodium, Aldrich, 2.50 g, 21.8 mmol, 10 eq.) under nitrogen. The resulting mixture was vigorously stirred at −78° C. for 9 h. The mixture was carefully poured into another flask. The remaining Na/Hg in the original flask was washed three times with DCM (10 mL×3). All the organics were combined, to the mixture was added sat. aq. solution of NH₄Cl (30 mL). The mixture was then extracted by DCM (200 mL followed by 30 mL×3). The combined organic phases were dried (Na₂SO₄), filtered, and concentrated in vacuo to give a crude product (TBDMS ether of the title compound), which was used for the next step immediately without any further purifications. MS (ES+): m/z=637.23/639.23 [MH⁺]. HPLC: t_{R}=2.04 min (polar_{—}3 min, TOF).

The crude material prepared above was dissolved in THF (40.0 mL) at 0° C., 4.0 M of HCl in H₂O (16.5 mL, 66.0 mmol, 30 eq.) was added at 0° C., and the resulting mixture was stirred at rt for 30-45 min. NaHCO₃ (45 eq.) was added to the mixture slowly at 0° C. to adjust pH to =9. Then the bulk of the solvent was removed under reduced pressure to give a residue, which was diluted by DCM (100 mL) and extracted by DCM (200 mL followed by 30 mL×3). The combined organic phases were dried (Na₂SO₄), filtered, and concentrated in vacuo to give a crude residue which was purified by silica gel chromatography (eluent: 30% EtOAc in DCM, then 2% MeOH in DCM to 5% MeOH in DCM) to give the title compound (70-75% yield, 2 steps). ¹H NMR (400 MHz, CD₃OD): δ=1.45-1.60 (m, 2H), 1.88-2.02 (m, 2H), 2.07-2.23 (m, 4H), 3.70 (tt, J=11.0, 4.2 Hz, 1H), 4.23 (tt, J=11.8, 3.9 Hz, 1H), 5.10-5.37 (m, 2H), 5.42-5.56 (m, 1H), 6.74 (t, J=73.5 Hz, 1H), 7.17-7.38 (m, 3H), 7.73 (d, J=0.5 Hz, 1H), 7.90 (d, J=1.8 Hz, 1H), 7.99 (d, J=0.5 Hz, 1H), 8.40 (d, J=1.8 Hz, 1H). MS (ES+): m/z=523.13/525.14 [MH⁺]. HPLC: t_{R}=1.35 min (polar_{—}3 min, TOF).

### 5-[1-(trans-4-{[tert-Butyl(dimethyl)silyl]oxy}cyclohexyl)-1H-pyrazol-4-yl]-3-{1-[2-chloro-6-(difluoromethoxy)-3-fluorophenyl]-2-fluoro-2,2-bis(phenylsulfonyl)ethyl}-1H-pyrrolo[2,3-b]pyridine

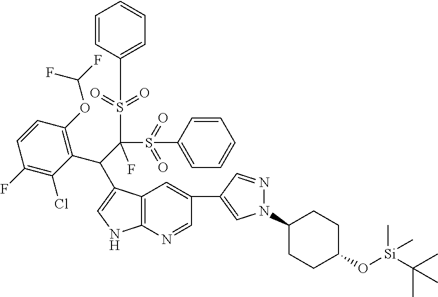 [SMILES: CC(C)(C)[Si](C)(C)O[C@H]1CC[C@H](n2cc(-c3cnc4[nH]cc(C(c5c(OC(F)F)ccc(F)c5Cl)C(F)(S(=O)(=O)c5ccccc5)S(=O)(=O)c5ccccc5)c4c3)cn2)CC1]

To a stirred mixture of 1-(trans-4-{[tert-butyl(dimethyl)silyl]oxy}cyclohexyl)-4-(4,4,5,5-tetramethyl-1,3,2-dioxaborolan-2-yl)-1H-pyrazole (Intermediate 4) (4.96 g, 9.78 mmol), 5-bromo-3-{1-[2-chloro-6-(difluoromethoxy)-3-fluorophenyl]-2-fluoro-2,2-bis(phenylsulfonyl)ethyl}-1H-pyrrolo[2,3-b]pyridine (5.20 g, 7.24 mmol) and potassium fluoride (1.47 g, 25.4 mmol) in 1,4-dioxane (80 mL) and H₂O (20 mL) was added (1,1′-bis-(diphenylphosphino)ferrocene)palladium dichloride (264 mg, 0.36 mmol) under nitrogen atmosphere. The resulting mixture was then stirred at 90° C. for 90 min. LC-MS indicated completion of reaction. Then the solvent was removed under reduced pressure to give a residue, which was purified by silica gel chromatography (eluent: from pure DCM to 20-30% EtOAc in DCM) to give desired product (82% yield). MS (ES+): m/z=917.20/919.20 [MH⁺]. HPLC: t_{R}=2.08 min (polar_{—}3 min, TOF).

### 5-Bromo-3-{1-[2-chloro-6-(difluoromethoxy)-3-fluorophenyl]-2-fluoro-2,2-bis(phenylsulfonyl)ethyl}-1H-pyrrolo[2,3-b]pyridine

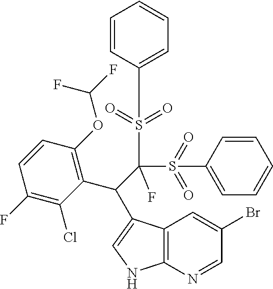 [SMILES: O=S(=O)(c1ccccc1)C(F)(C(c1c(OC(F)F)ccc(F)c1Cl)c1c[nH]c2ncc(Br)cc12)S(=O)(=O)c1ccccc1]

To a stirred solution of 1,1′-[(fluoromethanediyl)disulfonyl]dibenzene (26.3 g, 83.7 mmol) in THF (200 mL) was added 2.5 M of n-BuLi in hexane (32.4 mL, 80.9 mmol) at −78° C.; the resulting mixture was stirred for 30 min at −78° C. before use. To a stirred solution of 5-bromo-3-{[2-chloro-6-(difluoromethoxy)-3-fluorophenyl](methoxy)methyl}-1H-pyrrolo[2,3-b]pyridine (Intermediate 3) (12.15 g, 27.89 mmol) in anhydrous THF (120 mL) was added thionyl chloride (10.2 mL, 139 mmol) at rt. The resulting mixture was stirred for 90-120 min at 60° C., then the solvent and un-reacted thionyl chloride were distilled out under reduced pressure and the residue was dried under high vacuum for 1-2 hours. This residue was dissolved in anhydrous THF (200 mL) under nitrogen and cooled to −78° C. To this solution was then added the previously prepare mixture described above {2.5 M of n-BuLi and 1,1′-[(fluoromethanediyl)disulfonyl]dibenzene in THF at −78° C.} by cannula at −78° C. The resulting mixture was allowed to warm up to rt in about 2 h. The reaction was quenched by MeOH (10 mL) and sat. aq. NH₄Cl solution (50 mL). The bulk of solvent was removed under reduced pressure to give a residue, which was diluted by DCM (100 mL) and extracted by DCM (100 mL×3). The combined DCM layers were dried (Na₂SO₄), filtered, and concentrated in vacuo to give a crude residue which was purified by silica gel chromatography (eluent: from pure DCM to 10% EtOAc in DCM) to give the title compound (75% yield). MS (ES+): m/z=716.96/718.95/720.96 [MH⁺]. HPLC: t_{R}=1.53 min (polar_{—}3 min, TOF).

### Examples 4 & 5

### trans-4-[4-(3-{(1R)-1-[2-Chloro-6-(difluoromethoxy)-3-fluorophenyl]-2-fluoroethyl}-1H-pyrrolo[2,3-b]pyridin-5-yl)-1H-pyrazol-1-yl]cyclohexanol and trans-4-[4-(3-{(1S)-1-[2-Chloro-6-(difluoromethoxy)-3-fluorophenyl]-2-fluoroethyl}-1H-pyrrolo[2,3-b]pyridin-5-yl)-1H-pyrazol-1-yl]cyclohexanol

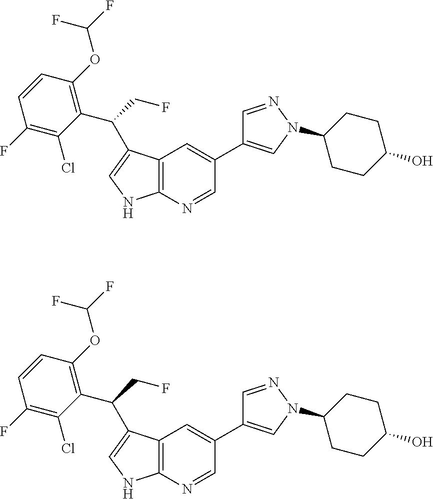 [SMILES: O[C@H]1CC[C@H](n2cc(-c3cnc4[nH]cc([C@@H](CF)c5c(OC(F)F)ccc(F)c5Cl)c4c3)cn2)CC1 ; O[C@H]1CC[C@H](n2cc(-c3cnc4[nH]cc([C@H](CF)c5c(OC(F)F)ccc(F)c5Cl)c4c3)cn2)CC1]

The racemic compound of Intermediate 6 was subjected to SFC separation on a chiral stationary phase to give two enantiomers. Preparative SFC (ChiralPak IA 21×250 mm I.D., solvent 60:40 scCO₂/isopropanol (0.2% isopropylamine) isocratic, flow rate 30 mL/min, UV detection at 254 nm): t_{R}=16.70 min [(1R) enantiomer=Example 4]; t_{R}=21.93 min [(1S) enantiomer=Example 5]. ¹HNMR and LC-MS data for both enantiomers are identical to the data obtained from the racemic mixture.

In further aspects, the compounds of the present invention include:

| | |
|---|---|
| | Ic |
| 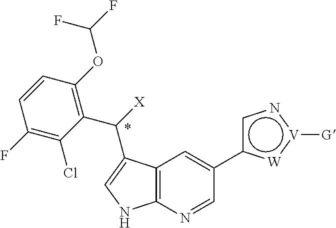 | |

| Example | | | | |
|---|---|---|---|---|
| No. | X | * | W—V | G¹ |
| 6 | CH₂F | R | CH—N | 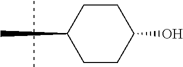 |
| 7 | CH₂F | R | CH—N | 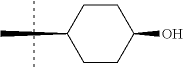 |
| 8 | CH₂F | R | CH—N | 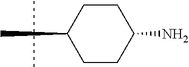 |
| 9 | CH₂F | R | CH—N | 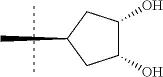 |
| 10 | CH₂F | R | CH—N | 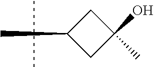 |
| 11 | CH₂F | R | CH—N | 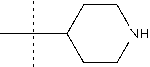 |
| 12 | CH₂F | R | CH—N | 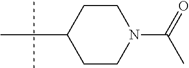 |
| 13 | CH₂F | S | CH—N | 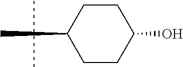 |
| 14 | CH₂F | S | CH—N | 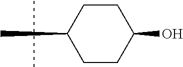 |
| 15 | CH₂F | S | CH—N | 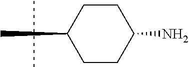 |
| 16 | CH₂F | S | CH—N | 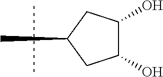 |
| 17 | CH₂F | S | CH—N | 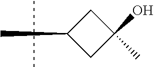 |
| 18 | CH₂F | S | CH—N | 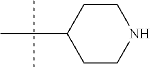 |
| 19 | CH₂F | S | CH—N | 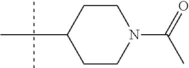 |
| 20 | CH₂F | R | NH—C | 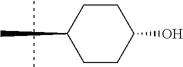 |
| 21 | CH₂F | R | NH—C | 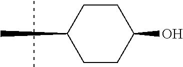 |
| 22 | CH₂F | R | NH—C | 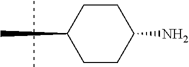 |
| 23 | CH₂F | R | NH—C | 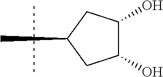 |
| 24 | CH₂F | R | NH—C | 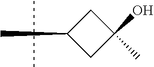 |
| 25 | CH₂F | R | NH—C | 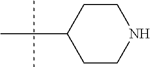 |
| 26 | CH₂F | R | NH—C | 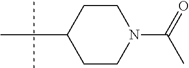 |
| 27 | CH₂F | R | NH—C | 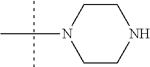 |
| 28 | CH₂F | R | NH—C | 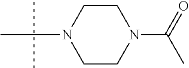 |
| 29 | CH₂F | R | NH—C | 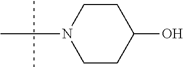 |
| 30 | CH₂F | S | NH—C | 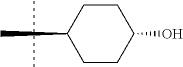 |
| 31 | CH₂F | S | NH—C | 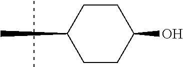 |
| 32 | CH₂F | S | NH—C | 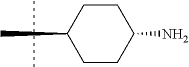 |
| 33 | CH₂F | S | NH—C | 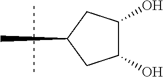 |
| 34 | CH₂F | S | NH—C | 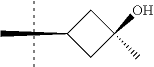 |
| 35 | CH₂F | S | NH—C | 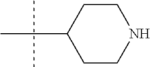 |
| 36 | CH₂F | S | NH—C | 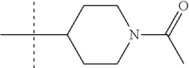 |
| 37 | CH₂F | S | NH—C | 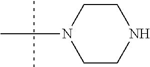 |
| 38 | CH₂F | S | NH—C | 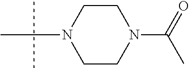 |
| 39 | CH₂F | S | NH—C | 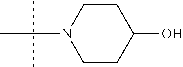 |
| 40 | CH₂F | R | S—C | 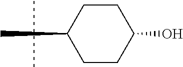 |
| 41 | CH₂F | R | S—C | 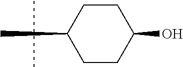 |
| 42 | CH₂F | R | S—C | 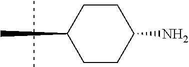 |
| 43 | CH₂F | R | S—C | 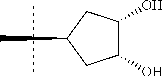 |
| 44 | CH₂F | R | S—C | 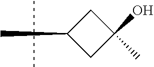 |
| 45 | CH₂F | R | S—C | 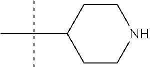 |
| 46 | CH₂F | R | S—C | 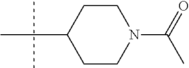 |
| 47 | CH₂F | R | S—C | 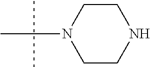 |
| 48 | CH₂F | R | S—C | 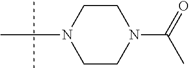 |
| 49 | CH₂F | R | S—C | 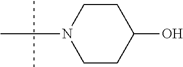 |
| 50 | CH₂F | S | S—C | 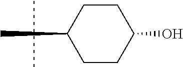 |
| 51 | CH₂F | S | S—C | 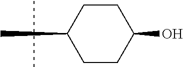 |
| 52 | CH₂F | S | S—C | 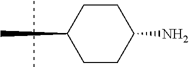 |
| 53 | CH₂F | S | S—C | 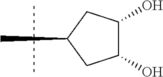 |
| 54 | CH₂F | S | S—C | 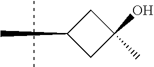 |
| 55 | CH₂F | S | S—C | 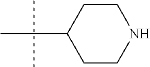 |
| 56 | CH₂F | S | S—C | 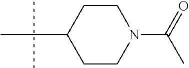 |
| 57 | CH₂F | S | S—C | 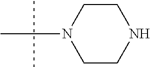 |
| 58 | CH₂F | S | S—C | 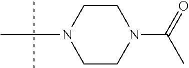 |
| 59 | CH₂F | S | S—C | 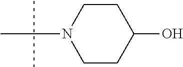 |

In further aspects, the compounds of the present invention also include:

| | |
|---|---|
| | Id |
| 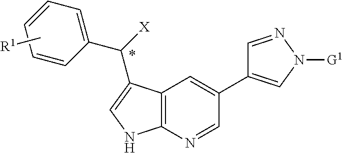 | |

| Ex- | | | | |
|---|---|---|---|---|
| am- | | | | |
| ple | | | | |
| No. | R¹ | X | * | G¹ |
| 60 | 2,6-di-Cl-3-F | CH₂F | R | 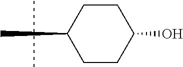 |
| 61 | 2,6-di-Cl-3-F | CH₂F | R | 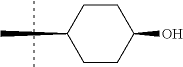 |
| 62 | 2,6-di-Cl-3-F | CH₂F | R | 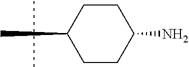 |
| 63 | 2,6-di-Cl-3-F | CH₂F | R | 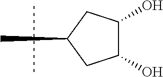 |
| 64 | 2,6-di-Cl-3-F | CH₂F | R | 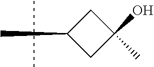 |
| 65 | 2,6-di-Cl-3-F | CH₂F | R | 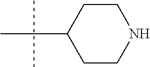 |
| 66 | 2,6-di-Cl-3-F | CH₂F | R | 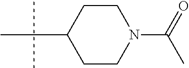 |
| 67 | 2,6-di-Cl-3-F | CH₂F | S | 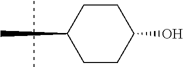 |
| 68 | 2,6-di-Cl-3-F | CH₂F | S | 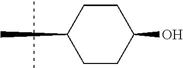 |
| 69 | 2,6-di-Cl-3-F | CH₂F | S | 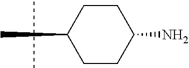 |
| 70 | 2,6-di-Cl-3-F | CH₂F | S | 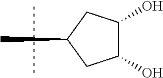 |
| 71 | 2,6-di-Cl-3-F | CH₂F | S | 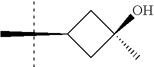 |
| 72 | 2,6-di-Cl-3-F | CH₂F | S | 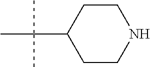 |
| 73 | 2,6-di-Cl-3-F | CH₂F | S | 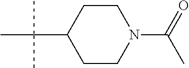 |
| 74 | 2-Cl-3-F-6-OCH₃ | CH₂F | R | 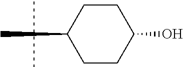 |
| 75 | 2-Cl-3-F-6-OCH₃ | CH₂F | R | 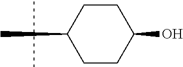 |
| 76 | 2-Cl-3-F-6-OCH₃ | CH₂F | R | 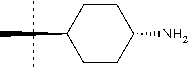 |
| 77 | 2-Cl-3-F-6-OCH₃ | CH₂F | R | 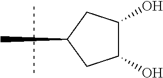 |
| 78 | 2-Cl-3-F-6-OCH₃ | CH₂F | R | 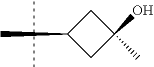 |
| 79 | 2-Cl-3-F-6-OCH₃ | CH₂F | R | 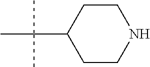 |
| 80 | 2-Cl-3-F-6-OCH₃ | CH₂F | R | 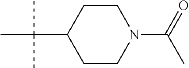 |
| 81 | 2-Cl-3-F-6-OCH₃ | CH₂F | S | 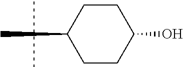 |
| 82 | 2-Cl-3-F-6-OCH₃ | CH₂F | S | 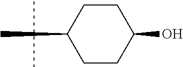 |
| 83 | 2-Cl-3-F-6-OCH₃ | CH₂F | S | 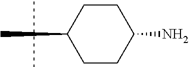 |
| 84 | 2-Cl-3-F-6-OCH₃ | CH₂F | S | 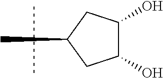 |
| 85 | 2-Cl-3-F-6-OCH₃ | CH₂F | S | 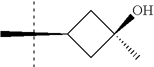 |
| 86 | 2-Cl-3-F-6-OCH₃ | CH₂F | S | 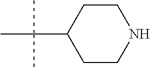 |
| 87 | 2-Cl-3-F-6-OCH₃ | CH₂F | S | 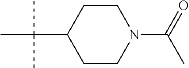 |
| 88 | 2,6-di-Cl- 3,5-di-OCH₃ | CH₂F | R | 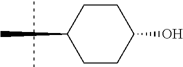 |
| 89 | 2,6-di-Cl- 3,5-di-OCH₃ | CH₂F | R | 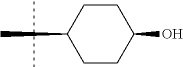 |
| 90 | 2,6-di-Cl- 3,5-di-OCH₃ | CH₂F | R | 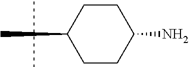 |
| 91 | 2,6-di-Cl- 3,5-di-OCH₃ | CH₂F | R | 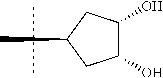 |
| 92 | 2,6-di-Cl- 3,5-di-OCH₃ | CH₂F | R | 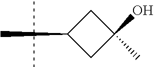 |
| 93 | 2,6-di-Cl- 3,5-di-OCH₃ | CH₂F | R | 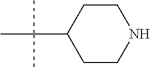 |
| 94 | 2,6-di-Cl- 3,5-di-OCH₃ | CH₂F | R | 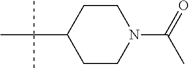 |
| 95 | 2,6-di-Cl- 3,5-di-OCH₃ | CH₂F | S | 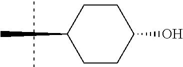 |
| 96 | 2,6-di-Cl- 3,5-di-OCH₃ | CH₂F | S | 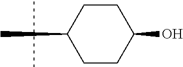 |
| 97 | 2,6-di-Cl- 3,5-di-OCH₃ | CH₂F | S | 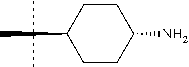 |
| 98 | 2,6-di-Cl- 3,5-di-OCH₃ | CH₂F | S | 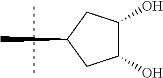 |
| 99 | 2,6-di-Cl- 3,5-di-OCH₃ | CH₂F | S | 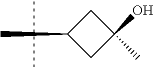 |
| 100 | 2,6-di-Cl- 3,5-di-OCH₃ | CH₂F | S | 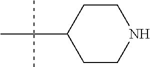 |
| 101 | 2,6-di-Cl- 3,5-di-OCH₃ | CH₂F | S | 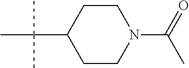 |

In further aspects, the compounds of the present invention also include:

| | |
|---|---|
| | Ie |
| 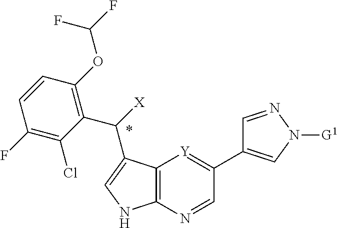 | |

| Example | | | | |
|---|---|---|---|---|
| No. | X | * | Y | G¹ |
| 102 | CH₂F | R | N | 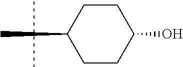 |
| 103 | CH₂F | R | N | 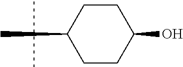 |
| 104 | CH₂F | R | N | 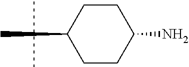 |
| 105 | CH₂F | R | N | 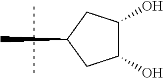 |
| 106 | CH₂F | R | N | 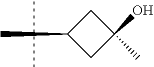 |
| 107 | CH₂F | R | N | 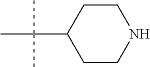 |
| 108 | CH₂F | R | N | 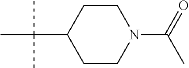 |
| 109 | CH₂F | S | N | 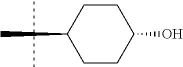 |
| 110 | CH₂F | S | N | 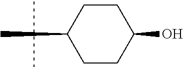 |
| 111 | CH₂F | S | N | 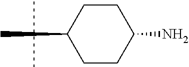 |
| 112 | CH₂F | S | N | 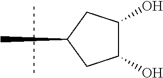 |
| 113 | CH₂F | S | N | 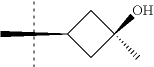 |
| 114 | CH₂F | S | N | 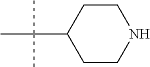 |
| 115 | CH₂F | S | N | 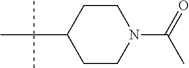 |
| 116 | CHF₂ | R | CH | 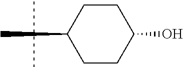 |
| 117 | CHF₂ | R | CH | 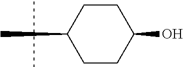 |
| 118 | CHF₂ | R | CH | 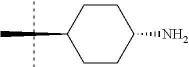 |
| 119 | CHF₂ | R | CH | 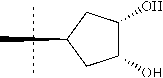 |
| 120 | CHF₂ | R | CH | 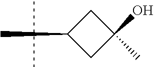 |
| 121 | CHF₂ | R | CH | 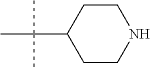 |
| 122 | CHF₂ | R | CH | 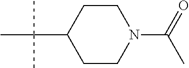 |
| 123 | CHF₂ | S | CH | 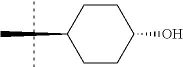 |
| 124 | CHF₂ | S | CH | 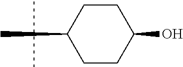 |
| 125 | CHF₂ | S | CH | 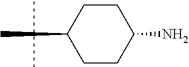 |
| 126 | CHF₂ | S | CH | 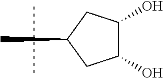 |
| 127 | CHF₂ | S | CH | 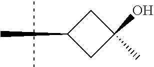 |
| 128 | CHF₂ | S | CH | 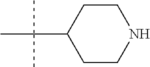 |
| 129 | CHF₂ | S | CH | 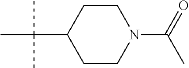 |
| 130 | CHF₂ | R | N | 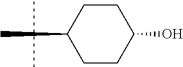 |
| 131 | CHF₂ | S | N | 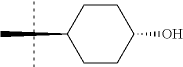 |
| 132 | CHF₂ | R | N | 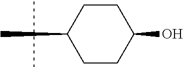 |
| 133 | CHF₂ | S | N | 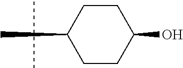 |
| 134 | CF₃ | R | CH | 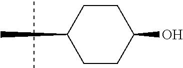 |
| 135 | CF₃ | S | CH | 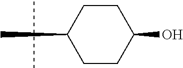 |
| 136 | CF₃ | R | CH | 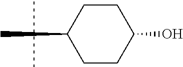 |
| 137 | CF₃ | S | CH | 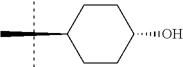 |

### Biological Data

The cellular activity of the compounds of the present invention against c-MET may be determined by the following procedure. MKN45 cells were plated in Falcon 3072 96-well plates in growth media (RPMI, 10% FBS, 1% L-glutamine) at a density of 5000 cells/well and incubated at 37° C., 5% CO₂ overnight. The following day, one-tenth volume of a 10× concentration of compounds was added to the wells in a 6-point dilution series. The dilutions series was composed of an initial 1:5 dilution in DMSO, followed by a 1:10 dilution in growth media, for a final DMSO concentration on cells of 0.5%. Control wells were treated with 0.5% DMSO. The typical range of dilution was 10 μM to 3 nM. Once compound was added to the cells, plates were incubated for 4 hours at 37° C., 5% CO₂. Plates were then washed in PBS, and lysed in triton-based lysis buffer. Lysates were transferred to a precoated capture plate made by Biosource (Cat #KHO0281). The phosphorylated MET levels were measured by incubating with a rabbit polyclonal antibody against phosphorylated MET ([pYpYpY1230/1234/1235]) followed by an anti-rabbit antibody conjugated to HRP. Signal was measured on a Wallac Victor plate reader at 450 nm. The DMSO signal of the control wells was defined as 100% and the percent of inhibition of phosphorylated MET was expressed as percent of control. IC₅₀ values were determined from the percent of control data using a standard four-parameter model.

The IC₅₀ values of exemplary compounds of the present invention determined in a MET cell mechanistic assay using the MKN45 cell line according to the procedures described herein in at least duplicate experiments are abbreviated as follows and are shown in Table 1: A, IC₅₀≦0.03 μM; B, 0.03 μM<IC₅₀≦0.1 μM; C, 0.1 μM<IC₅₀≦1 μM; D, 1 μM<IC₅₀≦3 μM; ND, not determined. The Example # of Table 1 corresponds to the compound Example number as illustrated in the Examples section.

TABLE 1
IC₅₀ values of examples in MET cell mechanistic assay (MKN45)

| | Example | | | | |
|---|---|---|---|---|---|
| | 1 | 2 | 3 | 4 | 5 |
| MET mech IC₅₀ | ND | ND | A | C | A |

The effect of inhibitors on the proliferation of MKN45 cells was determined using the following protocol. MKN45 cells were plated in Corning 3917 96-well white tissue culture treated plates in growth medium (RPMI, 10% FCS) at a density of 5000 cells/well in a total volume of 135 μL and incubated at 37° C., 5% CO₂, 95% humidity overnight. The following day, one-tenth volume of a 10× concentration of compounds was added to the wells in an 8-point dilution series. The dilution series was composed of an initial 1:5 dilution of a 10 mM stock of compound in DMSO, followed by serial 1:4 dilutions in DMSO, then a 1:20 dilution in growth medium prior to the 1:10 dilution into the cell plate. Final DMSO concentration on the cells was 0.1%, there were control wells treated with both 0.1% DMSO and no DMSO. The typical dilution range is 10 μM to 0.6 nM. Once the compound was added to the cells, plates were incubated for 3 days at 37° C., 5% CO₂ at 95% humidity. On the third day, after allowing all cells and reagents to come to room temperature, 25 μL of CellTiter-Glo reagent (Promega #G7573) was added to the wells. Plates were shaken on a platform for 10 minutes prior to reading luminescence for 0.1 seconds. The signal of the control wells was taken as 100% growth and growth inhibition was expressed as percent of control. IC₅₀ values were determined from the percent of control data using a standard four-parameter model.

The IC₅₀ values of exemplary compounds of the present invention determined in a cell proliferation assay using the MKN45 cell line according to the procedures described herein in at least duplicate experiments are abbreviated as follows and are shown in Table 2: A, IC₅₀≦0.03 μM; B, 0.03 μM<IC₅₀≦0.1 μM; C, 0.1 μM<IC₅₀≦1 μM; D, 1 μM<IC₅₀≦3 μM; ND, not determined. The Example # of Table 2 corresponds to the compound example number as illustrated in the Examples section.

MKN45 is a human gastric carcinoma cell line that shows a high level of amplification of c-MET and constitutive activation of c-MET. Treatment of this cell line with a selective c-MET inhibitor led to induction of apoptosis and inhibition of proliferation, whereas non-MET-amplified cell lines were not affected [Smolen et al., Proc. Natl. Acad. Sci. USA, 103(7):2316-2321 (2006)]. This cell line is thus “driven” by c-MET, and antiproliferative effects correlate very well with the inhibition of c-MET phosphorylation so that the cell proliferation IC₅₀ values can be used as surrogate for the c-MET cell mechanistic IC₅₀ values.

TABLE 2
IC₅₀ values of examples in MKN45 cell proliferation assay

| Example | 1 | 2 | 3 | 4 | 5 | 6 | 7 |
|---|---|---|---|---|---|---|---|
| Prolif. IC₅₀ | C | ND | C | A | ND | B | A |

The cellular activity of the compounds of the present invention against RON may be determined by the following procedure. HeLa cells were plated in Falcon 3072 96-well plates in growth media (DMEM, 10% FBS, 1% L-glutamine) at a density of 10000 cells/well and incubated at 37° C., 5% CO₂ overnight. The following day, cells were transfected with 0.2 μg sfRON-pcDNA plasmid DNA with 0.5 μL Lipofectamine2000 per well in the presence of 50 μL OPTI-MEM, incubated at 37° C., 5% CO₂ overnight. Costar 3915 96-well assay plates were coated with rabbit Anti-RON antibody at 2.0 μg/mL, sealed, and incubated overnight at 4° C. On the third day, coated plates were washed with PBS and blocked with 3% BSA. For the sfRON transfected cells, one-tenth volume of a 10× concentration of compounds was added to the wells in a 6-point dilution series. The dilution series was composed of an initial 1:5 dilution of a 10 mM DMSO stock solution of compound in DMSO, followed by a 1:10 dilution in growth media, for a final DMSO concentration on cells of 0.5%. Control wells were treated with 0.5% DMSO. The typical range of dilution was 10 μM to 3 nM. Once compound was added to the cells, plates were incubated for four hours at 37° C., 5% CO₂. Plates were then washed in PBS, and lysed in triton-based lysis buffer. Lysates were transferred to the blocked capture plates. The phosphorylated RON levels were measured by incubating with a Goat polyclonal antibody against phosphorylated RON ([pYpY1238/1239]) followed by an anti-Goat antibody conjugated to HRP. Signal was measured on a Wallac Victor plate reader with luminance. The DMSO signal of the control wells was defined as 100% and the percent of inhibition of phosphorylated RON was expressed as percent of control. IC₅₀ values were determined from the percent of control data using a standard four-parameter model.

The IC₅₀ values of exemplary compounds of the present invention determined in a sfRON cell mechanistic assay using the HeLa cell line according to the procedures described herein in at least duplicate experiments are abbreviated as follows and are shown in Table 3: A, IC₅₀≦0.03 μM; B, 0.03 μM<IC₅₀≦0.1 μM; C, 0.1 μM<IC₅₀≦1 μM; D, 1 μM<IC₅₀≦3 μM; ND, not determined. The Example # of Table 3 corresponds to the compound example number as illustrated in the Examples section.

TABLE 3
IC₅₀ values of examples in sfRON cell mechanistic assay (HeLa)

| | Example | | | | |
|---|---|---|---|---|---|
| | 1 | 2 | 3 | 4 | 5 |
| sfRON mech IC₅₀ | C | D | B | B | A |

The cellular activity of the compounds of the present invention against Aurora B may be determined by the following procedure. HT-29 cells grown in complete growth media (McCoy's 5A, 10% FCS, 1% L-glutamine) were plated into wells of a 96 well tissue culture plate (Falcon 3072) at a cell density of 4×10⁴ cells/0.09 ml media/well. Cells were subsequently incubated overnight in a 5% CO₂ humidified 37° C. incubator. The following day 10 μl of a 10× stock of test compound serially diluted in media was added to the cells and incubated for 1 h at 37° C. at which time Calyculin A (Cell Signaling #9902) was added at a concentration of 100 nM and cells incubated for an additional 30 minutes in a 5% CO₂ humidified 37° C. incubator. Media was then aspirated and cells lysed using a Triton based lysis buffer. Lysates were transferred to a pre-coated anti-Histone H3 antibody coated plate supplied by Cell Signaling in their PathScan phospho-Histone H3 (Ser10) ELISA kit (#7155). After an overnight incubation with lysate the ELISA was continued following the manufacturer's instructions. Signal was measured on a Wallac Victor plate reader at 450 nm. DMSO control treated cells served as 100% signal and an Aurora B kinase inhibitor served as 100% inhibition. The percent inhibition of phospho-Histone H3 (Ser10) was expressed as % control. IC₅₀ values were calculated from the percent control data using a standard four-parameter model.

The IC₅₀ values of exemplary compounds of the present invention determined in a Aurora B cell mechanistic assay using the HT-29 cell line according to the procedures described herein in at least duplicate experiments are abbreviated as follows and are shown in Table 4: A, IC₅₀≦0.03 μM; B, 0.03 μM<IC₅₀≦0.1 μM; C, 0.1 μM<IC₅₀≦1 μM; D, 1 μM<IC₅₀≦3 μM; ND, not determined. If only data from single experiments are available, the abbreviations are italicized. The Example # of Table 4 corresponds to the compound example number as illustrated in the Examples section.

TABLE 4
IC₅₀ values of examples in Aurora B cell mechanistic assay (HT-29)

| | Example | | | | |
|---|---|---|---|---|---|
| | 1 | 2 | 3 | 4 | 5 |
| Aurora B mech IC₅₀ | C | C | A | C | A |

The effect of inhibitors on the proliferation of Karpas-299 cells (DSMZ no. ACC 31) was determined using the following protocol. Karpas-299 cells were plated in 96-well white tissue culture treated plates (Corning 3917) in growth medium (RPMI, 10% FCS) at a density of 5000 cells/well in a total volume of 135 μL and incubated at 37° C., 5% CO₂, 95% humidity overnight. The following day, one-tenth volume of a 10× concentration of compounds was added to the wells in an 8-point dilution series. Compounds were serially diluted (1:4) in DMSO from a 10 mM stock solution prior to dilution in growth media to the 10× working concentrations (5% DMSO). Final concentration of DMSO in compound-treated wells was 0.5%. Control wells containing growth media or growth media/0.5% DMSO were included in all test plates. The typical dilution range is 10 μM to 0.1 nM. Once the compounds were added to the cells, plates were incubated for 3 days at 37° C., 5% CO₂ at 95% humidity. After 72 hours, all cells and reagents were equilibrated to room temperature and 15 μL of CellTiter-Glo reagent (Promega #G7573) was added to each well. Plates were shaken on a platform for 10 minutes at room temperature prior to reading luminescence. The value of the signal of the control wells was set as 100% growth and growth inhibition was expressed as percent of control. IC₅₀ values were determined from the percent of control data using a standard four-parameter curve fit equation.

The IC₅₀ values of exemplary compounds of the present invention determined in a cell proliferation assay using the Karpas-299 cell line according to the procedures described herein in at least duplicate experiments are abbreviated as follows and are shown in Table 5: A, IC₅₀≦0.03 μM; B, 0.03 μM<IC₅₀≦0.1 μM; C, 0.1 μM<IC₅₀≦1 μM; D, 1 μM<IC₅₀≦3 μM; ND, not determined. The Example # of Table 5 corresponds to the compound example number as illustrated in the Examples section.

The Karpas-299 cell line has a t(2;5) chromosomal translocation and expresses the NPM-ALK fusion protein, resulting in constitutively active ALK. A small-molecule ALK inhibitor inhibited growth of Karpas-299 cells at concentrations that showed a strong correlation to the inhibition of NPM-ALK total tyrosine phosphorylation [Christensen at al., Mol. Cancer. Ther. 6(12):3314-22 (2007)]. With this “ALK-driven” cell line by ALK, the cell proliferation IC₅₀ values can thus be used as surrogate for the p-ALK cell mechanistic IC₅₀ values.

TABLE 5
IC₅₀ values of examples in Karpas-299 cell proliferation assay

| | Example | | | | |
|---|---|---|---|---|---|
| | 1 | 2 | 3 | 4 | 5 |
| Prolif. IC₅₀ | C | C | B | C | A |

Compounds of Formula I (X═C₁₋₃haloaliphatic) show increased potency including against RON kinase with respect to comparator compounds that differ only in lacking the halogen (X═C₁₋₃aliphatic). Table 6 demonstrates this potency advantage. The Example numbers of Table 6 correspond to the compound example number as illustrated in the Examples section above. The IC₅₀ values shown in Table 6 are abbreviated as follows: A, IC₅₀≦0.03 μM; B, 0.03 μM<IC₅₀≦0.1 μM; C, 0.1 μM<IC₅₀≦1 μM; D, 1 μM<IC₅₀≦3 μM; E, IC₅₀≦3 μM.

TABLE 6
Comparison of IC₅₀ values of examples with X = C₁₋₃haloalkyl vs.
X = C₁₋₃alkyl

| | sfRON cell |
|---|---|
| Compound | mechanistic |
| Example 1 | C |
| 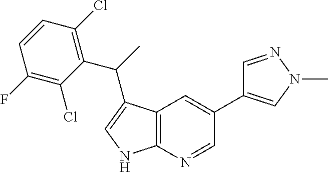 [SMILES: CC(c1c(Cl)ccc(F)c1Cl)c1c[nH]c2ncc(-c3cnn(C)c3)cc12] | D |
| Example 3 | B |
| 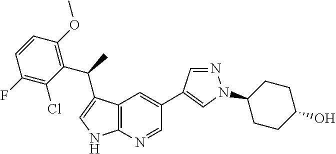 [SMILES: COc1ccc(F)c(Cl)c1[C@@H](C)c1c[nH]c2ncc(-c3cnn([C@H]4CC[C@H](O)CC4)c3)cc12] | C |
| Example 4 | B |
| 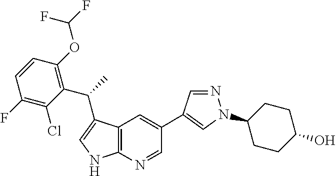 [SMILES: C[C@@H](c1c(OC(F)F)ccc(F)c1Cl)c1c[nH]c2ncc(-c3cnn([C@H]4CC[C@H](O)CC4)c3)cc12] | E |
| Example 5 | A |
| 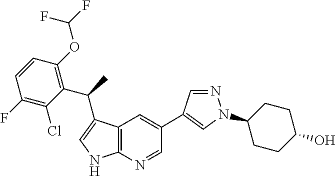 [SMILES: C[C@H](c1c(OC(F)F)ccc(F)c1Cl)c1c[nH]c2ncc(-c3cnn([C@H]4CC[C@H](O)CC4)c3)cc12] | B |

### Compositions

The invention includes pharmaceutical compositions comprising a compound or pharmaceutically acceptable salt thereof of the invention, which is formulated for a desired mode of administration with or without one or more pharmaceutically acceptable and useful carriers. The compounds can also be included in pharmaceutical compositions in combination with one or more other therapeutically active compounds.

The pharmaceutical compositions of the present invention comprise a compound of the invention (or a pharmaceutically acceptable salt thereof) as an active ingredient, optional pharmaceutically acceptable carrier(s) and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Compounds of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

### Uses

Compounds of the invention inhibit the activity of tyrosine kinase enzymes in animals, including humans, and are useful in the treatment and/or prevention of various diseases and conditions such as hyperproliferative disorders such as cancer. In particular, compounds disclosed herein are inhibitors of at least one of MET, RON, and ALK kinases.

In some aspects, compounds of the invention are useful as inhibitors of kinases, including in some aspects at least one of the MET, ALK, and RON kinases. In some aspects, compounds are active against IR and/or IGF-1R.

In some aspects, compounds of the invention are useful as inhibitors of kinases, including one or more of MET, RON, ALK, Trk, AXL, Tie-2, Flt3, FGFR3, Abl, Jak2, c-Src, IGF-1R, IR, PAK1, PAK2, and TAK1 kinases. In some aspects, compounds of the invention are inhibitors of kinases, including one or more of Blk, c-Raf, PRK2, Lck, Mek1, PDK-1, GSK3β, EGFR, p70S6K, BMX, SGK, CaMKII, and Tie-2 kinases.

In some aspects, compounds of the invention are useful as selective inhibitors of one or more of MET, RON, ALK, IGF-1R, or IR. In some embodiments, the compound is useful as a selective inhibitor of MET and/or RON and/or ALK over other kinase targets, such as KDR and/or Aurora kinase B (AKB). In some aspects, compounds of the invention are useful as selective inhibitors of MET, RON, ALK with selectivity over KDR and Aurora kinase B (AKB).

In some aspects, compounds of the invention are useful in treating proliferative disease, particularly cancers, including cancers, including cancers mediated or driven by one or more of MET, RON, ALK, IR, or IGF-1R, or other target(s), or cancers for which inhibition of such targets is useful, alone or in combination with other active agents.

In some aspects, compounds of the invention are useful as selective inhibitors of one or more of MET, RON, and ALK with selectivity over AKB and/or KDR of at least about 2, 4, 8, 10, 16, 20, 32, 40-fold, or greater.

In some aspects, the invention includes a method of treating cancer, tumors, and tumor metastases, comprising administering to a mammal in need thereof a therapeutically effective amount of a compound or salt of the invention.

In some aspects, the invention includes a method of treating a cancer mediated at least in part by RON and/or MET comprising administering to a mammal in need thereof a therapeutically effective amount of a compound or salt of Formula I.

In some aspects, the invention includes a method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of Formula I.

The compounds of Formula I of the present invention are useful in the treatment of a variety of cancers, including, but not limited to, solid tumor, sarcoma, fibrosarcoma, osteoma, melanoma, retinoblastoma, rhabdomyosarcoma, glioblastoma, neuroblastoma, teratocarcinoma, hematopoietic malignancy, and malignant ascites. More specifically, the cancers include, but not limited to, lung cancer, bladder cancer, pancreatic cancer, kidney cancer, gastric cancer, breast cancer, colon cancer, prostate cancer (including bone metastases), hepatocellular carcinoma, ovarian cancer, esophageal squamous cell carcinoma, melanoma, an anaplastic large cell lymphoma, an inflammatory myofibroblastic tumor, and a glioblastoma.

In some aspects, the above methods are used to treat one or more of bladder, colorectal, nonsmall cell lung, breast, or pancreatic cancer. In some aspects, the above methods are used to treat one or more of ovarian, gastric, head and neck, prostate, hepatocellular, renal, glioma, glioma, or sarcoma cancer.

In some aspects thereof, at least one additional anti-cancer agent is administered in a therapeutically effective combination regimen. In some aspects thereof, the additional agent comprises an agent that acts on a biological target involved in compensatory signaling or cross-talk with at least one of RON, MET, or ALK. In some aspects thereof, the agents in the combination regimen behave synergistically. In some aspects thereof, the at least one additional anti-cancer agent comprises a VEGF, IGF-1R, or EGFR inhibitor.

In some aspects, the invention includes a method of treating cancer comprising administering to a mammal in need thereof a therapeutically effective amount of a compound or salt of the invention, wherein at least one additional active anti-cancer agent is used as part of the method. In some aspects, the additional agent(s) is an EGFR inhibitor and/or an IGF-1R inhibitor.

In some aspects, the invention includes a method, including the above methods, wherein the compound is used to inhibit EMT (Epithelial Mesenchymal Transition).

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In some aspects, the invention includes a method of treating cancer comprising administering to a mammal in need thereof a therapeutically effective amount of a compound or salt of the invention, wherein at least one additional active anti-cancer agent is used as part of the method.

### General Definitions and Abbreviations

Except where otherwise indicated, the following general conventions and definitions apply. Unless otherwise indicated herein, language and terms are to be given their broadest reasonable interpretation as understood by the skilled artisan. Any examples given are nonlimiting.

Any section headings or subheadings herein are for the reader's convenience and/or formal compliance and are non-limiting.

A recitation of a compound herein is open to and embraces any material or composition containing the recited compound (e.g., a composition containing a racemic mixture, tautomers, epimers, stereoisomers, impure mixtures, etc.). In that a salt, solvate, or hydrate, polymorph, or other complex of a compound includes the compound itself, a recitation of a compound embraces materials containing such forms. Isotopically labeled compounds are also encompassed except where specifically excluded. For example, hydrogen is not limited to hydrogen containing zero neutrons.

The term “active agent” of the invention means a compound of the invention in any salt, polymorph, crystal, solvate, or hydrated form.

The term “pharmaceutically acceptable salt(s)” is known in the art and includes salts of acidic or basic groups which can be present in the compounds and prepared or resulting from pharmaceutically acceptable bases or acids.

The term “substituted” and substitutions contained in formulas herein refer to the replacement of one or more hydrogen radicals in a given structure with a specified radical, or, if not specified, to the replacement with any chemically feasible radical. When more than one position in a given structure can be substituted with more than one substituent selected from specified groups, the substituents can be either the same or different at every position (independently selected) unless otherwise indicated. In some cases, two positions in a given structure can be substituted with one shared substituent. It is understood that chemically impossible or highly unstable configurations are not desired or intended, as the skilled artisan would appreciate.

In descriptions and claims where subject matter (e.g., substitution at a given molecular position) is recited as being selected from a group of possibilities, the recitation is specifically intended to include any subset of the recited group. In the case of multiple variable positions or substituents, any combination of group or variable subsets is also contemplated.

Unless indicated otherwise, a substituent, diradical or other group referred to herein can be bonded through any suitable position to a referenced subject molecule. For example, the term “indolyl” includes 1-indolyl, 2-indolyl, 3-indolyl, etc.

The convention for describing the carbon content of certain moieties is “(Cₐ-b)” or “Cₐ-C_{b}” meaning that the moiety can contain any number of from “a” to “b” carbon atoms. C₀alkyl means a single covalent chemical bond when it is a connecting moiety, and a hydrogen when it is a terminal moiety. Similarly, “x-y” can indicate a moiety containing from x to y atoms, e.g., ₅₋₆heterocycloalkyl means a heterocycloalkyl having either five or six ring members. “C_{x-y}” may be used to define number of carbons in a group. For example, “C₀₋₁₂alkyl” means alkyl having 0-12 carbons, wherein C₀alkyl means a single covalent chemical bond when a linking group and means hydrogen when a terminal group.

The term “absent,” as used herein to describe a structural variable (e.g., “—R— is absent”) means that diradical R has no atoms, and merely represents a bond between other adjoining atoms, unless otherwise indicated.

Unless otherwise indicated (such as by a connecting “-”), the connections of compound name moieties are at the rightmost recited moiety. That is, the substituent name starts with a terminal moiety, continues with any bridging moieties, and ends with the connecting moiety. For example, “heteroarylthioC₁₋₄alkyl is a heteroaryl group connected through a thio sulfur to a C₁₋₄ alkyl, which alkyl connects to the chemical species bearing the substituent.

The term “aliphatic” means any hydrocarbon moiety, and can contain linear, branched, and cyclic parts, and can be saturated or unsaturated. The term includes, e.g., alkyl, alkenyl, alkynyl, cycloalkyl, carbocyclic, and others.

The term “alkyl” means any saturated hydrocarbon group that is straight-chain or branched. Examples of alkyl groups include methyl, ethyl, propyl, 2-propyl, n-butyl, iso-butyl, tert-butyl, pentyl, and the like.

The term “alkenyl” means any ethylenically unsaturated straight-chain or branched hydrocarbon group. Representative examples include, but are not limited to, ethenyl, 1-propenyl, 2-propenyl, 1-butenyl, 2-butenyl, or 3-butenyl, and the like.

The term “alkynyl” means any acetylenically unsaturated straight-chain or branched hydrocarbon group. Representative examples include, but are not limited to, ethynyl, 1-propynyl, 2-propynyl, 1-butynyl, 2-butynyl, or 3-butynyl, and the like.

The term “alkoxy” means —O-alkyl, —O-alkenyl, or —O-alkynyl. “Haloalkoxy” means an —O-(haloalkyl) group. Representative examples include, but are not limited to, trifluoromethoxy, tribromomethoxy, and the like.

“Haloalkyl” means an alkyl, preferably lower alkyl, that is substituted with one or more same or different halo atoms.

“Hydroxyalkyl” means an alkyl, preferably lower alkyl, that is substituted with one, two, or three hydroxy groups; e.g., hydroxymethyl, 1-hydroxyethyl or 2-hydroxyethyl, 1,2-dihydroxypropyl, 1,3-dihydroxypropyl, or 2,3-dihydroxypropyl, and the like.

The term “alkanoyl” means —C(O)-alkyl, —C(O)-alkenyl, or —C(O)-alkynyl.

“Alkylthio” means an (alkyl)-S— or a (unsubstituted cycloalkyl)-S— group. Representative examples include, but are not limited to, methylthio, ethylthio, propylthio, butylthio, cyclopropylthio, cyclobutylthio, cyclopentylthio, cyclohexylthio, and the like.

The term “cyclic” means any ring system with or without heteroatoms (N, O, or S(O)₀₋₂), and which can be saturated or unsaturated. Ring systems can be bridged and can include fused rings. The size of ring systems may be described using terminology such as “_{x-y}cyclic,” which means a cyclic ring system that can have from x to y ring atoms. For example, the term “₉₋₁₀carbocyclic” means a 5, 6 or 6,6 fused bicyclic carbocyclic ring system which can be satd., unsatd. or aromatic. It also means a phenyl fused to one 5 or 6 membered satd. or unsatd. carbocyclic group. Nonlimiting examples of such groups include naphthyl, 1,2,3,4-tetrahydronaphthyl, indenyl, indanyl, and the like.

The term “carbocyclic” means a cyclic ring moiety containing only carbon atoms in the ring(s) without regard to aromaticity. A 3-10 membered carbocyclic means chemically feasible monocyclic and fused bicyclic carbocyclics having from 3 to 10 ring atoms. Similarly, a 4-6 membered carbocyclic means monocyclic carbocyclic ring moieties having 4 to 6 ring carbons, and a 9-10 membered carbocyclic means fused bicyclic carbocyclic ring moieties having 9 to 10 ring carbons.

The term “cycloalkyl” means a non-aromatic 3-12 carbon mono-cyclic, bicyclic, or polycyclic aliphatic ring moiety. Cycloalkyl can be bicycloalkyl, polycycloalkyl, bridged, or spiroalkyl. One or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like.

The term “unsaturated carbocyclic” means any cycloalkyl containing at least one double or triple bond. The term “cycloalkenyl” means a cycloalkyl having at least one double bond in the ring moiety.

The terms “bicycloalkyl” and “polycycloalkyl” mean a structure consisting of two or more cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1]heptyl (“norbornyl”), bicyclo[2.2.2]octyl, and the like.

The term “spiroalkyl” means a structure consisting of two cycloalkyl moieties that have exactly one atom in common. Examples include, but are not limited to, spiro[4.5]decyl, spiro[2.3]hexyl, and the like.

The term “aromatic” means a planar ring moieties containing 4n+2 pi electrons, wherein n is an integer.

The term “aryl” means aromatic moieties containing only carbon atoms in its ring system. Non-limiting examples include phenyl, naphthyl, and anthracenyl. The terms “aryl-alkyl” or “arylalkyl” or “aralkyl” refer to any alkyl that forms a bridging portion with a terminal aryl.

“Aralkyl” means alkyl, preferably lower alkyl, that is substituted with an aryl group as defined above; e.g., phenylCH₂—, phenyl(CH₂)₂—, phenyl(CH₂)₃—, phenylCH₂(CH₃)CHCH₂—, and the like and derivatives thereof.

The term “heterocyclic” means a cyclic ring moiety containing at least one heteroatom (N, O, or S(O)₀₋₂), including heteroaryl, heterocycloalkyl, including unsaturated heterocyclic rings.

The term “heterocycloalkyl” means a non-aromatic monocyclic, bicyclic, or polycyclic heterocyclic ring moiety of 3 to 12 ring atoms containing at least one ring having one or more heteroatoms. The rings may also have one or more double bonds. However, the rings do not have a completely conjugated pi-electron system. Examples of heterocycloalkyl rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine, and the like. Other examples of heterocycloalkyl rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocycloalkyl rings. The term “heterocycloalkyl” also includes fused ring systems and can include a carbocyclic ring that is partially or fully unsaturated, such as a benzene ring, to form benzofused heterocycloalkyl rings. For example, 3,4-dihydro-1,4-benzodioxine, tetrahydroquinoline, tetrahydroisoquinoline, and the like. The term “heterocycloalkyl” also includes heterobicycloalkyl, heteropolycycloalkyl, or heterospiroalkyl, which are bicycloalkyl, polycycloalkyl, or spiroalkyl, in which one or more carbon atom(s) are replaced by one or more heteroatoms selected from O, N, and S. For example, 2-oxa-spiro[3.3]heptane, 2,7-diaza-spiro[4.5]decane, 6-oxa-2-thia-spiro[3.4]octane, octahydropyrrolo[1,2-a]pyrazine, 7-aza-bicyclo[2.2.1]heptane, 2-oxa-bicyclo[2.2.2]octane, 8-azabicyclo[3.2.1]octyl, bicyclo[3.1.0]hexyl, spiro[3.3]hept-2-yl, 2-azaspiro[3.3]hept-6-yl, 2-azaspiro[3.3]hept-2-yl, 2,7-diazaspiro[3.5]non-7-yl, and the like, are such heterocycloalkyls.

Examples of saturated heterocyclic groups include, but are not limited to oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl, and 1,4-diazepanyl.

Non-aryl heterocyclic groups include saturated and unsaturated systems and can include groups having only 4 atoms in their ring system. The heterocyclic groups include benzo-fused ring systems and ring systems substituted with one or more oxo moieties. Recitation of ring sulfur is understood to include the sulfide, sulfoxide or sulfone where feasible. The heterocyclic groups also include partially unsaturated or fully saturated 4-10 membered ring systems, e.g., single rings of 4 to 8 atoms in size and bicyclic ring systems, including aromatic 6-membered aryl or heteroaryl rings fused to a non-aromatic ring. Also included are 4-6 membered ring systems (“4-6 membered heterocyclic”), which include 5-6 membered heteroaryls, and include groups such as azetidinyl and piperidinyl. Heterocyclics can be heteroatom-attached where such is possible. For instance, a group derived from pyrrole can be pyrrol-1-yl (N-attached) or pyrrol-3-yl (C-attached). Other heterocyclics include imidazo[4,5-b]pyridin-3-yl and benzoimidazol-1-yl.

Examples of heterocyclic groups include pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, and the like.

The term “unsaturated heterocyclic” means a heterocycloalkyl containing at least one unsaturated bond. The term “heterobicycloalkyl” means a bicycloalkyl structure in which at least one carbon atom is replaced with a heteroatom. The term “heterospiroalkyl” means a spiroalkyl structure in which at least one carbon atom is replaced with a heteroatom.

Examples of partially unsaturated heteroalicyclic groups include, but are not limited to: 3,4-dihydro-2H-pyranyl, 5,6-dihydro-2H-pyranyl, 2H-pyranyl, 1,2,3,4-tetrahydropyridinyl, and 1,2,5,6-tetrahydropyridinyl.

The terms “heteroaryl” or “hetaryl” mean a monocyclic, bicyclic, or polycyclic aromatic heterocyclic ring moiety containing 5-12 atoms. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, and the like. Furthermore, the terms “heteroaryl” include fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

Heteroaryls include, e.g., 5- and 6-membered monocyclics such as pyrazinyl and pyridinyl, and 9- and 10-membered fused bicyclic ring moieties, such as quinolinyl. Other examples of heteroaryl include quinolin-4-yl, 7-methoxy-quinolin-4-yl, pyridin-4-yl, pyridin-3-yl, and pyridin-2-yl. Other examples of heteroaryl include pyridinyl, imidazolyl, pyrimidinyl, pyrazolyl, triazolyl, pyrazinyl, tetrazolyl, furanyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. Examples of 5-6 membered heteroaryls include, thiophenyl, isoxazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl, 1,3,5-triazinyl, and the like.

Examples of monocyclic heteroaryl groups include, but are not limited to: pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Examples of fused ring heteroaryl groups include, but are not limited to: benzoduranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, azaquinazoline, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrimido[2,3-b]pyrazinyl, and pyrimido[4,5-d]pyrimidinyl.

The term “Heteroaralkyl” group means alkyl, preferably lower alkyl, that is substituted with a heteroaryl group; e.g., pyridinylCH₂—, pyrimidinyl(CH₂)₂—, imidazolyl(CH₂)₃—, and the like, and derivatives thereof.

“Arylthio” means an arylS— or and heteroarylS— group, as defined herein. Representative examples include, but are not limited to, phenylthio, pyridinylthio, furanylthio, thienylthio, pyrimidinylthio, and the like and derivatives thereof.

The term “9-10 membered heterocyclic” means a fused 5, 6 or 6,6 bicyclic heterocyclic ring moiety, which can be satd., unsatd. or aromatic. The term “9-10 membered fused bicyclic heterocyclic” also means a phenyl fused to one 5 or 6 membered heterocyclic group. Examples include benzofuranyl, benzothiophenyl, indolyl, benzoxazolyl, 3H-imidazo[4,5-c]pyridin-yl, dihydrophthazinyl, 1H-imidazo[4,5-c]pyridin-1-yl, imidazo[4,5-b]pyridyl, 1,3 benzo[1,3]dioxolyl, 2H-chromanyl, isochromanyl, 5-oxo-2,3 dihydro-5H-[1,3]thiazolo[3,2-a]pyrimidyl, 1,3-benzothiazolyl, 1,4,5,6 tetrahydropyridazyl, 1,2,3,4,7,8 hexahydropteridinyl, 2-thioxo-2,3,6,9-tetrahydro-1H-purin-8-yl, 3,7-dihydro-1H-purin-8-yl, 3,4-dihydropyrimidin-1-yl, 2,3-dihydro-1,4-benzodioxinyl, benzo[1,3]dioxolyl, 2H-chromenyl, chromanyl, 3,4-dihydrophthalazinyl, 2,3-dihydro-1H-indolyl, 1,3-dihydro-2H-isoindol-2-yl, 2,4,7-trioxo-1,2,3,4,7,8-hexahydropteridin-yl, thieno[3,2-d]pyrimidinyl, 4-oxo-4,7-dihydro-3H-pyrrolo[2,3-d]pyrimidin-yl, 1,3-dimethyl-6-oxo-2-thioxo-2,3,6,9-tetrahydro-1H-purinyl, 1,2-dihydroisoquinolinyl, 2-oxo-1,3-benzoxazolyl, 2,3-dihydro-5H-1,3-thiazolo-[3,2-a]pyrimidinyl, 5,6,7,8-tetrahydro-quinazolinyl, 4-oxochromanyl, 1,3-benzothiazolyl, benzimidazolyl, benzotriazolyl, purinyl, furylpyridyl, thiophenylpyrimidyl, thiophenylpyridyl, pyrrolylpiridyl, oxazolylpyridyl, thiazolylpiridyl, 3,4-dihydropyrimidin-1-yl imidazolylpyridyl, quinoliyl, isoquinolinyl, quinazolinyl, quinoxalinyl, naphthyridinyl, pyrazolyl[3,4]pyridine, 1,2-dihydroisoquinolinyl, cinnolinyl, 2,3-dihydro-benzo[1,4]dioxin-4-yl, 4,5,6,7-tetrahydro-benzo[b]-thiophenyl-2-yl, 1,8-naphthyridinyl, 1,5-napthyridinyl, 1,6-naphthyridinyl, 1,7-napthyridinyl, 3,4-dihydro-2H-1,4-benzothiazine, 4,8-dihydroxy-quinolinyl, 1-oxo-1,2-dihydro-isoquinolinyl, 4-phenyl-[1,2,3]thiadiazolyl, and the like.

“Aryloxy” means an arylO— or a heteroarylO— group, as defined herein. Representative examples include, but are not limited to, phenoxy, pyridinyloxy, furanyloxy, thienyloxy, pyrimidinyloxy, pyrazinyloxy, and the like, and derivatives thereof.

One in the art understands that an “oxo” requires a second bond from the atom to which the oxo is attached. Accordingly, it is understood that oxo cannot be subststituted onto an aryl or heteroaryl ring.

The term “halo” means fluoro, chloro, bromo, or iodo.

“Acyl” means a —C(O)R group, where R can be selected from the nonlimiting group of hydrogen or optionally substituted lower alkyl, trihalomethyl, unsubstituted cycloalkyl, aryl. “Thioacyl” or “thiocarbonyl” means a —C(S)R″ group, with R as defined above.

The term “protecting group” means a suitable chemical group that can be attached to a functional group and removed at a later stage to reveal the intact functional group. Examples of suitable protecting groups for various functional groups are described in T. W. Greene and P. G. M. Wuts, Protective Groups in Organic Synthesis, 2d Ed., John Wiley and Sons (1991 and later editions); L. Fieser and M. Fieser, Fieser and Fieser's Reagents for Organic Synthesis, John Wiley and Sons (1994); and L. Paquette, ed. Encyclopedia of Reagents for Organic Synthesis, John Wiley and Sons (1995). The term “hydroxy protecting group”, as used herein, unless otherwise indicated, includes Ac, CBZ, and various hydroxy protecting groups familiar to those skilled in the art including the groups referred to in Greene.

As used herein, the term “pharmaceutically acceptable salt” means those salts which retain the biological effectiveness and properties of the parent compound and do not present insurmountable safety or toxicity issues.

The term “pharmaceutical composition” means an active compound in any form suitable for effective administration to a subject, e.g., a mixture of the compound and at least one pharmaceutically acceptable carrier.

As used herein, a “physiologically/pharmaceutically acceptable carrier” means a carrier or diluent that does not cause significant irritation to an organism and does not abrogate the biological activity and properties of the administered compound.

A “pharmaceutically acceptable excipient” means an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The terms “treat,” “treatment,” and “treating” means reversing, alleviating, inhibiting the progress of, or partially or completely preventing the disorder or condition to which such term applies, or one or more symptoms of such disorder or condition. “Preventing” means treating before an infection occurs.

“Therapeutically effective amount” means that amount of the compound being administered which will relieve to some extent one or more of the symptoms of the disorder being treated, or result in inhibition of the progress or at least partial reversal of the condition.

The following abbreviations are used:

min. minute(s)

h hour(s)

d day(s)

RT or rt room temperature

t_{R} retention time

L liter

mL milliliter

mmol millimole

μmol micromole

equiv. or eq. equivalents

NMR nuclear magnetic resonance

MDP(S) mass-directed HPLC purification (system)

LC/MS liquid chromatography mass spectrometry

HPLC high performance liquid chromatography

TLC thin layer chromatography

CDCl₃ deuterated chloroform

CD₃OD or MeOD deuterated methanol

DMSO-d₆ deuterated dimethylsulfoxide

LDA lithium diisopropylamide

DCM dichloromethane

THF tetrahydrofuran

EtOAc ethyl acetate

MeCN acetonitrile

DMSO dimethylsulfoxide

Boc tert-butyloxycarbonyl

DME 1,2-dimethoxyethane

DMF N,N-dimethylformamide

DIPEA diisopropylethylamine

PS-DIEA polymer-supported diisopropylethylamine

PS-PPh₃-Pd polymer-supported Pd(PPh₃)₄

EDC 1-(3-dimethylaminopropyl)-3-ethylcarbodiimide

HOBt 1-hydroxybenzotriazole

DMAP 4-dimethylaminopyridine

TBTU O-(benzotriazol-1-yl)-N,N,N′,N′-tetramethyluronium tetrafluoroborate

TEMPO 2,2,6,6-tetramethylpiperidine-1-oxyl

TFA trifluoroacetic acid

What is claimed is:

1. A compound of Formula I: 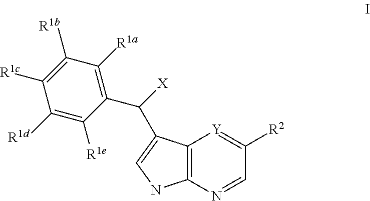
or a pharmaceutically acceptable salt thereof, wherein:
Y is CH or N;
X is C₁₋₃haloaliphatic;
R^{1a}, R^{1b}, R^{1c}, R^{1d}, and R^{1e} are each independently selected from H, halogen, —CN, C₁₋₆aliphatic, —OC₀₋₆aliphatic, —S(O)ₘC₁₋₆aliphatic, —SO₂N(C₀₋₆aliphatic)(C₀₋₆aliphatic), —N(C₀₋₆aliphatic)(C₀₋₆aliphatic), —N(C₀₋₆aliphatic)C(═O)C₀₋₆aliphatic, —N(C₀₋₆aliphatic)C(═O)OC₀₋₆aliphatic, —N(C₀₋₆aliphatic)C(═O)N(C₀₋₆aliphatic)(C₀₋₆aliphatic), —C(═O)C₀₋₆aliphatic, —C(═O)OC₀₋₆aliphatic, —C(═O)N(C₀₋₆aliphatic)(C₀₋₆aliphatic), —N(C₀₋₆aliphatic)-heterocyclyl, —N(C₀₋₆aliphatic)-heteroaryl, C₃₋₈cycloaliphatic, —O-cyclic, —O-heterocyclyl, sulfide, sulfoxide, or —S-cyclic, any of which is optionally substituted with one or more halogen, —CN, —OC₀₋₆aliphatic, —N(C₀₋₆aliphatic)(C₀₋₆aliphatic), —C(═O)N(C₀₋₆aliphatic)(C₀₋₆aliphatic), —C(═O)OC₀₋₆aliphatic, —C(═O)C₀₋₆aliphatic, heterocyclyl, or heteroaryl;
or heterocyclyl, which is optionally substituted with oxo, C₁₋₆aliphatic, C(═O)OC₁₋₆aliphatic, C(═O)C₀₋₆aliphatic, C(═O)N(C₀₋₆aliphatic)(C₀₋₆aliphatic), SO₂N(C₀₋₆aliphatic)(C₀₋₆aliphatic), SO₂(C₁₋₆aliphatic), heteroaryl, —S-heteroaryl, or —O-heteroaryl;
R² is selected from H, halo, —CN, —CF₃, —NO₂, C₀₋₆aliphatic, C₃₋₆cycloaliphaticC₀₋₆aliphatic, 3-6 membered heterocycloalkylC₀₋₆aliphatic, 3-6 membered heterocycloalkenylC₀₋₆aliphatic, arylC₀₋₆aliphatic, or heteroarylC₀₋₆aliphatic, any of which is optionally substituted with one or more G¹;
each G¹ is independently 4-10 membered heterocycloalkyl or heteroaryl optionally substituted with one or more OH, —CN, —OR⁶, R⁶, halogen, oxo, —NR⁶R⁷, —S(O)ₘR⁶, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —P(O)R^{a}R^{b}, —P(O)(R^{a})OR⁶, —P(O)(OR⁶)(OR⁷) or C₁₋₆alkyl, which is optionally substituted by halogen or —OC₀₋₅alkyl;
or G¹ is ₃₋₈cycloalkyl optionally substituted with one or more OH, —CN, —OR⁶, R⁶, halogen, oxo, —NR⁶R⁷, —S(O)ₘR⁶, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —P(O)R^{a}R^{b}, —P(O)(R^{a})OR⁶, —P(O)(OR⁶)(OR⁷) or —C₁₋₆alkyl which alkyl can be substituted by halogen or —OC₀₋₅alkyl;
or G¹ is C₁₋₆aliphatic optionally substituted with one or more —OH, —CN, —OR⁶, R⁶, halogen, oxo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, —P(O)R^{a}R^{b}, —P(O)(R^{a})OR⁶, —P(O)(OR⁶)(OR⁷), or 4-7 membered heterocycloalkyl optionally substituted by C₁₋₆alkyl;
wherein each R⁶, R⁷, R⁸, R⁹, R¹⁰, R^{a}, and R^{b} is independently C₀₋₅alkyl, C₃₋₆cycloalkyl, or 4-8 membered heterocycloalkyl optionally substituted with halogen, —OCF₃, or —OC₀₋₃alkyl;
or —NR⁶R⁷ is 4-7 membered heterocycloalkyl optionally substituted with C₁₋₆alkyl;
or R⁸ and R⁹, R^{a} and R^{b}, R^{a} and OR⁶, or OR⁶ and OR⁷, taken together can combine with the atom that they are attached to form a 4-8 membered heterocycloalkyl or C₃₋₈cycloalkyl ring optionally substituted by C₁₋₆alkyl;
n is independently 0-7; and
m is independently 0-2.

2. The compound or salt of claim 1, wherein:
Y is CH;
X is C₁₋₂haloalkyl; and
R² is selected from C₃₋₆cycloalkylC₀₋₆alkyl, 3-6 membered heterocycloalkylC₀₋₆alkyl, 3-6 membered heterocycloalkenylC₀₋₆alkyl, arylC₀₋₆alkyl, or heteroarylC₀₋₆alkyl, any of which is optionally substituted with 1-3 G¹.

3. The compound or salt of claim 1, wherein:
Y is CH;
X is halomethyl; and
R² is a 5-membered heteroaryl which can be independently substituted with 1-2 G¹.

4. The compound or salt of claim 3, wherein:
R² is

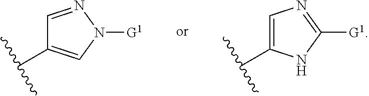

5. The compound or salt of claim 4, wherein:
R^{1a} and R^{1e} are each independently selected from halogen, —CN, C₁₋₃alkyl, —OC₀₋₃alkyl, wherein alkyl can be independently substituted with 1-3 fluorine atoms; and
R^{1b}, R^{1c}, and R^{1d} are each independently selected from H, halogen, —CN, C₁₋₃alkyl, —OC₀₋₃alkyl, wherein alkyl can be independently substituted with 1-3 fluorine atoms, —OC₀₋₆alkyl, —N(C₀₋₆alkyl)(C₀₋₆alkyl), —C(═O)N(C₀₋₆alkyl)(C₀₋₆alkyl), —C(═O)OC₀₋₆alkyl, —C(═O)C₀₋₆alkyl, or 5-6 membered heteroaryl.

6. The compound or salt of claim 5, wherein:
G¹ is C₁₋₆alkyl substituted with 0-3 substituents independently selected from OH, —CN, —OR⁶, —C(O)R^{b}, —C(O)NR⁶R⁷, —C(O)C(O)NR⁶R⁷, —C(O)OR⁶, —C(O)C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, —P(O)R^{a}R^{b}, —P(O)(R^{a})OR⁶, —P(O)(OR⁶)(OR⁷), or 4-7 membered heterocycloalkyl optionally substituted with C₁₋₆alkyl;
wherein each R⁶, R⁷, R⁸, R⁹, R¹⁰, R^{a}, and R^{b} are independently C₀₋₅alkyl or C₃₋₇cycloalkyl, each independently optionally substituted with halogen, —OCF₃, or —OC₀₋₃alkyl.

7. The compound or salt of claim 5, wherein:
G¹ is 4-8 membered heterocycloalkyl substituted with 0-3 substituents independently selected from OH, —CN, —OR⁶, halogen, R⁶, —S(O)ₘR⁶, —SO₂NR⁶R⁷, —C(O)R^{b}, —C(O)NR⁶R⁷, —C(O)C(O)NR⁶R⁷, —C(O)OR⁶, —C(O)C(O)OR⁶, —P(O)R^{a}R^{b}, —P(O)(R^{a})OR⁶, or —P(O)(OR⁶)(OR⁷);
or G¹ is C₃₋₈cycloalkyl substituted with 0-3 substituents independently selected from OH, —CN, —OR⁶, halogen, —S(O)ₘR⁶, —SO₂NR⁶R⁷, —C(O)R^{b}, —C(O)NR⁶R⁷, —C(O)C(O)NR⁶R⁷, —C(O)OR⁶, —C(O)C(O)OR⁶, —P(O)R^{a}R^{b}, —P(O)(R^{a})OR⁶, —P(O)(OR⁶)(OR⁷), or C₁₋₆alkyl optionally substituted with halogen or —OC₀₋₅alkyl;
wherein each R⁶, R⁷, R^{a}, and R^{b} is independently C₀₋₅alkyl or C₃₋₇cycloalkyl.

8. The compound or salt of claim 7, wherein:
R^{1b} and R^{1d} are each independently selected from H, halogen, —CN, C₁₋₃alkyl, or —OC₁₋₃alkyl, wherein alkyl can be substituted with 1-3 fluorine atoms; and
R^{1c} is H.

9. The compound or salt of claim 8, wherein:
G¹ is C₃₋₈cycloalkyl substituted with 0-3 substituents independently selected from OH, —CN, —OR⁶, halogen, —S(O)ₘR⁶, —SO₂NR⁶R⁷, —C(O)R^{b}, —C(O)NR⁶R⁷, —C(O)OR⁶, —P(O)R^{a}R^{b}, —P(O)(R^{a})OR⁶, —P(O)(OR⁶)(OR⁷), or C₁₋₆alkyl optionally substituted with halogen or —OC₀₋₅alkyl;
wherein each R⁶, R⁷, R^{a}, and R^{b} is independently C₀₋₅alkyl or C3-cycloalkyl.

10. The compound or salt of claim 8, wherein:
G¹ is 4-8 membered heterocycloalkyl substituted with 0-3 substituents independently selected from OH, —CN, —OR⁶, halogen, R⁶, —S(O)ₘR⁶, —SO₂NR⁶R⁷, —C(O)R^{b}, —C(O)NR⁶R⁷, —C(O)OR⁶, —P(O)R^{a}R^{b}, —P(O)(R^{a})OR⁶, or —P(O)(OR⁶)(OR⁷).

11. The compound or salt of claim 10, wherein:
R^{1a} is halogen, or methoxy optionally substituted with 1-3 fluorine atoms; and
R^{1d} and R^{1e} are independently halogen.

12. The compound or salt of claim 11, wherein
G¹ is 4-7 membered heterocycloalkyl optionally substituted with one or more independent halogen, —OH, —OCH₃, or C₁₋₃alkyl.

13. The compound or salt of claim 12, wherein:
G¹ is C₄₋₇cycloalkyl optionally substituted with one or more independent halogen, —OH, —OCH₃, or C₁₋₃alkyl.

14. The compound or salt of claim 13, wherein:
G¹ is cyclohexanol;
R^{1a} is —OCHF₂;
R^{1d} is fluoro; and
R^{1e} is chloro.

15. (canceled)

16. The compound or salt of claim 3, which is present as a material that is substantially free of its (R)-1-(phenyl) haloethyl enantiomer.

17. The compound or salt of claim 3, which is present as a material that is substantially free of its (S)-1-(phenyl)haloethyl enantiomer.

18. The compound or salt of claim 1, which exhibits inhibition of c-Met in a cellular mechanistic assay with an IC₅₀ of about 50 nM or less.

19-20. (canceled)

21. The compound or salt of claim 1, selected from any one of Examples 1-137 herein.

22-23. (canceled)

24. A method of treating a cancer mediated at least in part by RON and/or MET comprising administering to a mammal in need thereof a therapeutically effective amount of a compound or salt of claim 1.

25. A method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of claim 1.

26-29. (canceled)
